(19) United States
(12) Patent Application Publication
(10) Pub. No.: US 2024/0423461 A1
(43) Pub. Date: Dec. 26, 2024

(54) MEDICAL INSTRUMENTS

(71) Applicant: TYMPANY MEDICAL LIMITED, Galway (IE)

(72) Inventors: Rory O'Callaghan, Cavan (IE); Elizabeth McGloughlin, Galway (IE); Jibran Ahmed Abbasi, Galway (IE); Neil Clancy, Kildare (IE); Michael Lynch, Limerick (IE); Thomas Gillen, Bad Säckingen (DE); Cesare Stefanini, Pisa (IT)

(21) Appl. No.: 18/274,108

(22) PCT Filed: Jan. 25, 2022
(86) PCT No.: PCT/EP2022/051656

§ 371 Date: Jul. 25, 2023

(30) Foreign Application Priority Data
Jan. 25, 2021 (EP) 21153353.4
Dec. 13, 2021 (EP) 21214195.6

(51) Int. Cl. A61B 1/045; A61B 1/00; A61B 1/06; A61B 1/227

(52) U.S. Cl. CPC A61B 1/045; A61B 1/00013; A61B 1/00029; A61B 1/0646; A61B 1/0661; A61B 1/227

(57) ABSTRACT

An imaging head of a medical scope comprises a capsule that encapsulates an image sensor and electronics associated with the image sensor. The imaging head further comprises a light guide, within or attached to the capsule, that conveys illumination to a field of view of the image sensor. The imaging head can be disassembled by breaking or cutting the capsule to separate the image sensor and the electronics from the light guide. The imaging head is manufactured by encapsulating the image sensor and electronics, and optionally also the light guide, in a capsule. The light guide may be overmoulded in a first overmoulding step to embed the light guide in an overmoulded body. The image sensor and electronics may then be positioned beside or against the overmoulded body and overmoulded in a second overmoulding step to complete the capsule.

This invention relates to medical instruments, exemplified in this specification by medical scopes such as endoscopes or otoscopes for use in examining and treating the ears.

Instruments such as endoscopes and otoscopes may be employed to examine and perform surgery on ears. Some such instruments may be held against the surgeon's eye to view and magnify the subject whereas other such instruments have a camera that allows the subject to be viewed on a video display such as a computer monitor.

Effective access and visualisation are key to safe surgical procedures. In this respect, traditional ear surgery employing microscopic visualisation suffers from the disadvantage of a narrow field of view looking down into the ear canal. As a result, there can be poor visualisation of disease in the middle ear in regions that are difficult to access, such as the sinus tympani and facial recess. Whilst trans-canal surgery may be performed through a speculum by using a microscope and specialised surgical instruments, this again suffers from a narrow field of view.

Thus, to improve the field of view, it is often necessary to expose the middle ear and attic area by performing a mastoidectomy. However, as the ears are surrounded by dense bone, mastoidectomy procedures are associated with increased operating times, potentially significant complications, longer hospital stays and protracted recovery.

Another disadvantage of microscopic visualisation in ear surgery is the substantial size of a surgical microscope. This compromises the surgeon's dexterity, restricts the space available for surgical intervention and may have negative ergonomic consequences for the surgeon when using the microscope.

Endoscopic ear surgery has become a growing clinical speciality, not least because of the drawbacks of microscopic visualisation. In comparison to a surgical microscope, an endoscope offers improved visibility and allows less invasive surgery. However, the ear presents particular challenges to the use of an endoscope, with a requirement for the image-sensing part of the endoscope to be as small as possible while maintaining image quality and allowing multiple angles of view. Another challenge relates to cleaning, especially to keeping a lens of the endoscope clean in use, and more generally to cleaning or reconditioning the endoscope for reuse.

In use, tissue or other contaminants adhering to the lens of an endoscope can obscure vision. Consequently, the endoscope may have to be removed from a subject's ear canal repeatedly during surgery for cleaning. This interrupts and prolongs the surgical procedure and increases the risk of errors or inadvertent injury to the patient.

In relation to reuse, there is a risk that an endoscope could be contaminated with prions that may be present in the ear canal. Contamination with the wrong type of prions could endanger a patient's life. Even cleaning and sterilisation processes used in industry cannot remove certain types of contamination such as prions; in this respect, prions cannot be killed as they are not living organisms. Currently, therefore, the World Health Organisation recommends that all instruments used in surgery are discarded after surgery and so are single use wherever possible.

In view of this and other risks, single-use endoscopes for ear surgery have appeared on the market. Indeed, there is now a significant trend for hospitals and manufacturers to move from a model of reuse and maintenance of endoscopic equipment to single-use endoscopy. This trend has been driven to some extent by the inability of in-hospital sterilisation to sterilise equipment fully, hence reducing confidence in the system.

Unlike simple single-use hospital equipment, such as scalpel blades and syringes, single-use endoscopes are large and generate significant amounts of medical waste. Medical waste is typically incinerated, rendering materials irrecoverable. In the case of instruments containing electronics, this can include precious metals or rare earth metals. The result is a waste of valuable resources and potentially a negative impact on the environment.

At first sight, moving from a single-use system to a circular economy system could be seen as a solution to this problem. For a manufacturer of single-use instruments, moving to a circular economy system would firstly involve recovering used instruments from a hospital in a reverse logistics operation. This recovery step would be followed by: dismantling the instruments; identifying components that can be reused and cleaning them; discarding components that cannot be reused and replacing them with new components; assembling a new instrument from the reused and new components; and packing and sterilising the new instrument ready for distribution. However, this process is not feasible as an alternative to single-use endoscopy without incorporating special design features in the endoscope and in the processes used in the circular economy system.

The invention addresses how a surgical instrument may be designed for integration into a circular economy or reverse logistics system in which at least a substantial portion of the instrument can be reused safely and cost-effectively, with minimal waste. The inventive concept also embraces a method by which a surgical instrument can be reconditioned for reuse under a circular economy or reverse logistics system.

More generally, the invention also improves prior solutions to provide a panoramic endoscope for ear surgery that is compact, reliable and easy to keep clean in use and that provides highly-effective visualisation.

Embodiments of the invention describe how a camera of an endoscope can be integrated into a rotating head to satisfy the requirement of enabling multiple angles of view. Ideally, the rotating head can turn through 360°. This makes it convenient to clean the lens of the camera by rotating the head and hence the lens past a useful viewing angle and over a wiper blade that removes debris from the lens. Elegantly, this allows one simple mechanism to solve two of the major challenges of endoscopic ear surgery.

To allow an endoscope to operate in this way, the camera unit of the rotating head is an integrated module that incorporates a lens, an image sensor and data transmission provisions. Data may be transmitted wirelessly from the rotating head to a supporting probe structure of the endoscope or to a control unit outside the endoscope. A wireless solution has the advantage of negating wired data connections that could otherwise limit rotation of the head.

Electrical power must also be delivered to the rotating head to power the camera module and any other electrical systems such as illumination LEDs, which commonly are found alongside chip-on-tip image sensors in endoscope designs. Again, this should ideally be done in a way that does not limit rotation of the head.

One solution for delivering electrical power to a rotating head involves the use of rotating or sliding contacts on or around the axis of rotation, where the head is joined to the supporting probe of the endoscope. This requires a robust sealing arrangement to insulate the live contacts from moisture in the ear and from saline solution used in surgery, which is electrically conductive. Another solution is to deliver power wirelessly or electromagnetically, for example in a manner akin to an inductive charging unit. However, implementing either of these solutions remains challenging due to the compactness that is required of an endoscope for ear surgery.

The narrowness of the ear canal determines that, optimally, the outer diameter of the probe and the rotating head should be no greater than 3.2 mm. Wireless power delivery requires a coil in the rotating head that, in this context, could significantly restrict the space available for other components in the head, notably the camera module itself.

The same size constraints also have a significant impact on image quality as they dictate that the image sensor of the camera must be small in area, typically smaller than 2 mm×2 mm. This in turn means a small pixel size; and with higher resolution, the pixel size gets smaller still. A problem then ensues because smaller sensor pixels are less sensitive to light than larger pixels. This results in a darker and lower-quality image than would be delivered by a larger sensor, all other things being equal. To counteract this, a larger optical aperture or lens can be used to capture more light but this has the knock-on effect of a reduced depth of field. Indeed, in sufficiently compact optical systems, the depth of field can become so restricted as to be unusable for surgery.

Ultimately, what is needed to offset the small size of the system is better illumination. This can require the provision of bigger and more powerful LEDs or the use of a remote light source and optical fibres or other light transmission features.

Against this background, the invention resides in a medical scope comprising an imaging head, wherein the imaging head comprises: a capsule that encapsulates an image sensor and electronics associated with the image sensor, and a light guide arranged to convey illumination to a field of view of the image sensor. The light guide may be within the capsule or may be attached to the capsule.

The imaging head may further include a disassembly interface at which the encapsulated image sensor and electronics are preferentially separable from the light guide. For example, the disassembly interface may comprise at least one point or line of weakness in or on the imaging head or the capsule. Such a feature may be aligned with a joint of the imaging head disposed between the light guide and the image sensor and electronics. The disassembly interface may further comprise a protective barrier embedded within the capsule.

A probe of the scope may comprise a mount at which the imaging head is movably attached to the probe. The imaging head may be cantilevered from the mount. Elegantly, the capsule or the light guide may include a formation that is shaped to engage movably with the mount. The light guide is preferably disposed between the mount and the image sensor in a direction parallel to an axis of rotation about which the imaging head can turn relative to the mount.

The imaging head is suitably disposed at a distal end of the probe. A handle may be separably mounted to a proximal end of the probe. The handle may comprise a drive for moving the imaging head relative to the probe; a light source for conveying illumination along the probe to the light guide of the imaging head; and power and/or data connections for conveying power and/or data along the probe to and/or from the imaging head. Thus, a drive interface, an optical interface, and power and/or data interfaces are suitable disposed between the handle and the probe. The probe may also support a protective over-sheath that can be pulled over the handle.

The inventive concept embraces a corresponding method of disassembling an imaging head of a medical scope. The method then comprises: breaking or cutting a capsule of the imaging head that encapsulates an image sensor and associated electronics; and separating the image sensor and the electronics from a light guide of the imaging head that is arranged to convey illumination to a field of view of the image sensor. In a preliminary step, the capsule is preferably checked for sealing integrity before being broken, cut or otherwise penetrated.

The light guide may be separated from the capsule, for example by being removed from within the capsule or by being detached from the exterior of the capsule. Force may be applied to a line or point of weakness of the imaging head or the capsule to separate the image sensor and the electronics from the light guide.

After use, the scope may be placed into a container that is then sealed around the scope. Conveniently, before use, the scope may have been removed from the same container in a sterile state. Before sealing, water may be added to the container to activate a cleaning agent in the container. The sealed container may then be placed in a depository, which can be conveyed to a disassembly unit when full or nearly full.

When the container is received at the disassembly unit, whether directly or via a depository, the scope may be unpacked from the container and then disassembled into parts. Unpacking and/or disassembly preferably take place robotically. During disassembly, a partial vacuum may be applied to the scope or a gas flow may be applied across the scope. This prevents contaminant particles transferring from the disassembled exterior to the interior components during disassembly. The parts may be sorted into: parts for recycling; parts for reuse, which may include the image sensor; waste for destruction; and/or biodegradable materials. The parts for reuse may then be shipped to a remanufacturing facility.

The inventive concept also extends to a method of manufacturing an imaging head of a medical scope of the invention. The method comprises encapsulating an image sensor and associated electronics of the imaging head in a capsule, the imaging head further comprising a light guide arranged to convey illumination to a field of view of the image sensor.

The light guide may also be encapsulated in the capsule. For example, the light guide may be overmoulded in a first overmoulding step to embed the light guide in an overmoulded body. The image sensor and electronics may then be positioned against or beside the overmoulded body before being overmoulded in a second overmoulding step to complete the capsule. An induction coil may also be embedded in the overmoulded body.

The overmoulded body may be formed on a mount about which the imaging head is movable relative to a probe of the scope. For example, the mount may be a formation about which the imaging head can rotate relative to the probe.

Thus, in the invention, an imaging head of a medical scope comprises a capsule that encapsulates an image sensor and electronics associated with the image sensor. The imaging head further comprises a light guide, within or attached to the capsule, that conveys illumination to a field of view of the image sensor. The imaging head can be disassembled by breaking or cutting the capsule to separate the image sensor and the electronics from the light guide.

The imaging head is manufactured by encapsulating the image sensor and electronics, and optionally also the light guide, in a capsule. The light guide may be overmoulded in a first overmoulding step to embed the light guide in an overmoulded body. The image sensor and electronics may then be positioned beside or against the overmoulded body and overmoulded in a second overmoulding step to complete the capsule.

In another aspect of the invention, a medical scope comprises: an elongate body or probe; an imaging head supported by and movable relative to a mount of the body, the imaging head including a light emitter for illuminating a field of view of the imaging head; and a light path extending from the body and through the imaging head to the light emitter, the light path including a light inlet of the imaging head opposed to a light outlet of the body. The imaging head is preferably cantilevered from the mount.

Conveniently, the light inlet and the light outlet may be mutually opposed across a mounting interface, such as a pivot coupling at which the imaging head is movably attached to the mount.

Where the imaging head contains an image sensor, the imaging head may further comprise a photovoltaic generator arranged such that impingement of light generates electrical power for the image sensor. The imaging head may further comprise a data transmitter powered by the photovoltaic generator to transmit image data from the image sensor to a receiver outside the imaging head.

The imaging head may also comprise a light guide in the light path, configured to direct a portion of light received from the body onto the photovoltaic generator and another portion of light received from the body to the light emitter. The light guide may, for example, comprise a filter that is configured to divide the light received from the body into said portions to be directed onto the photovoltaic generator and toward the light emitter.

Where the light inlet and the light outlet are disposed on an axis of rotation about which the imaging head can turn relative to the mount, the light guide may be disposed between the mount and the image sensor in a direction parallel to that axis. The light emitter may also be disposed between the mount and the image sensor in a direction parallel to the axis of rotation, at a position spaced radially from the axis of rotation.

The imaging head may comprise encapsulation around the image sensor. That encapsulation may include a disassembly interface at which the image sensor is preferentially separable from the light emitter. The disassembly interface may, for example, comprise at least one point of weakness in the encapsulation. The disassembly interface may also, or instead, comprise a protective strip embedded along a cutting line in the encapsulation.

The inventive concept also embraces a corresponding method of illuminating a field of view of an imaging head of a medical scope, which head is movable relative to a supporting body of the scope. The method comprises conveying light along a light path from a light outlet of the body to a light inlet of the imaging head and then through the imaging head to be emitted from the imaging head.

Elegantly, as noted above, an image sensor of the imaging head may be powered with electricity generated within the imaging head from at least a portion of the light conveyed along the light path.

Light travelling along the light path within the imaging head may be filtered to separate that light into components for power generation and for illumination, respectively. At least one of those components of the light may be varied, for example in intensity, relative to another of those components of the light to enable independent adjustment of power generation and/or illumination.

Embodiments of the invention deliver light longitudinally through a supporting structure to a rotating endoscope head. Light is used as a mechanism for power delivery to a chip-on-tip endoscope for ear surgery.

In some embodiments, sealed capsules are used to protect components designed for disassembly in a circular economy system. Provision may be made for daisy-chaining for transmission of light, power or data between the capsules.

In another aspect of the invention, a medical scope comprises: an imaging head supported by and movable relative to a body such as a probe, the imaging head including an image sensor and a light emitter for illuminating a field of view of the image sensor; a light path extending from the body to the light emitter, the light path including a light inlet of the imaging head opposed to a light outlet of the body across a wireless interface between the imaging head and the body; and a light transmitter in the imaging head, the light transmitter being coupled to the light path to transmit image data from the image sensor to the body across the interface.

Elegantly, the light inlet and the light outlet may be mutually opposed across a mount at which the imaging head is movably attached to the body. For example, the light inlet and the light outlet may be disposed on an axis of rotation about which the imaging head can turn relative to the body. In a compact arrangement, the light emitter may be disposed between the interface and the image sensor in a direction parallel to the axis of rotation, at a position spaced radially from the axis of rotation.

The interface may further comprise a wireless power transmission coupling effecting transmission of power from the body to the image sensor. The power transmission coupling may, for example, comprise mutually-opposed coils, a first of those coils surrounding the light inlet of the imaging head and a second of those coils surrounding the light outlet of the body. In an alternative approach, the imaging head may include a photovoltaic generator for powering the image sensor from light transmitted across the interface.

The imaging head may further comprise a filter in the light path, configured to block reflected light from the light emitter and to pass light transmitted from the light transmitter.

A light source outside the imaging head may be coupled to the light path to provide illuminating light to the light emitter. In that case, the light source and the image sensor may be synchronised such that the light source is on when the image sensor is capturing an image frame and the light source is off between image frames while the light transmitter transmits imaging data from the image sensor.

An image sensor controller outside the imaging head may be arranged to convey a control signal from the image sensor controller across the interface to the image sensor. The control signal may comprise a clock signal multiplexed with a write signal.

The image sensor controller may, for example, be connected to the wireless power transmission coupling of the interface to convey the control signal to the image sensor through that coupling. The image sensor controller could instead drive an LED to convey the control signal to the image sensor as an optical control signal. In that case, the light transmitter in the imaging head may be responsive to the optical control signal.

The inventive concept embraces a corresponding method of operating a scope that comprises an imaging head movable relative to a supporting body. The method comprises: illuminating a field of view of the imaging head by conveying light along a light path across a wireless interface from the body to the imaging head; and transmitting image data optically along the light path across the interface from the imaging head to the body.

In order that the invention may be more readily understood, reference will now be made, by way of example, to the accompanying drawings in which:

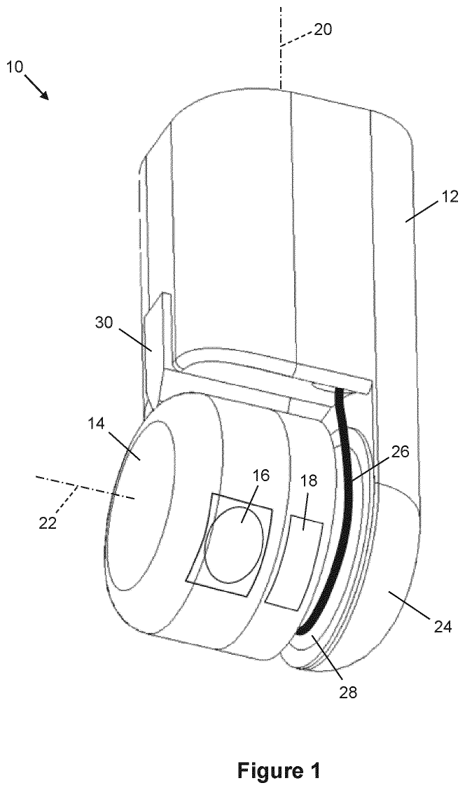

FIG. 1 is a perspective view of a distal end portion of an endoscope of the invention;

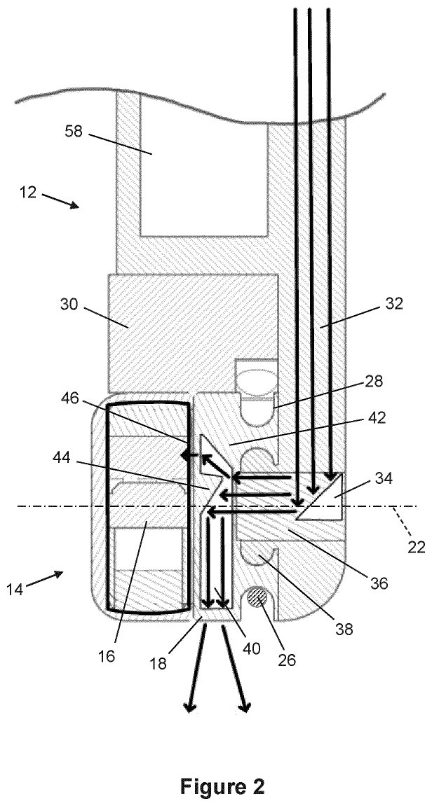

FIG. 2 is a side view of the endoscope of FIG. 1 in longitudinal section;

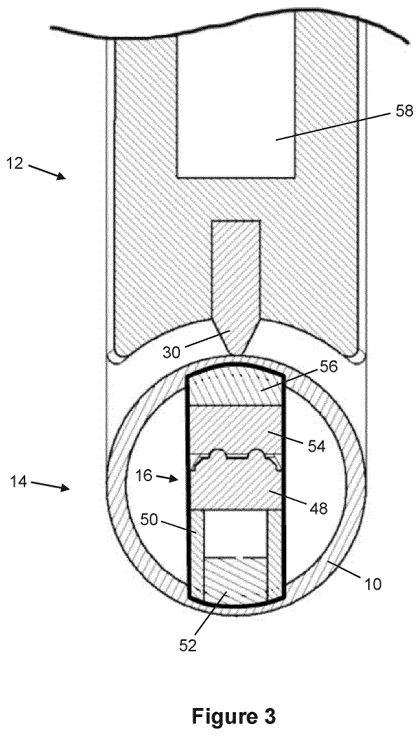

FIG. 3 corresponds to FIG. 2 but shows the endoscope from a direction orthogonal to that shown in FIG. 2 about a longitudinal axis;

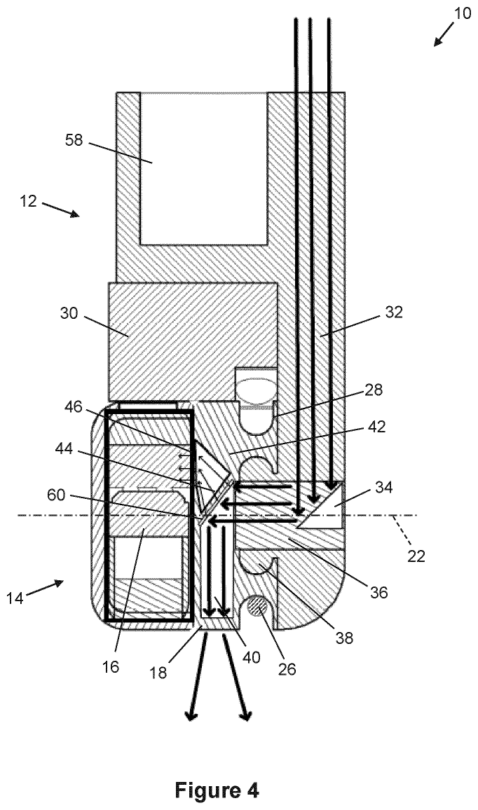

FIG. 4 shows a variant of the endoscope but otherwise corresponds to FIG. 2;

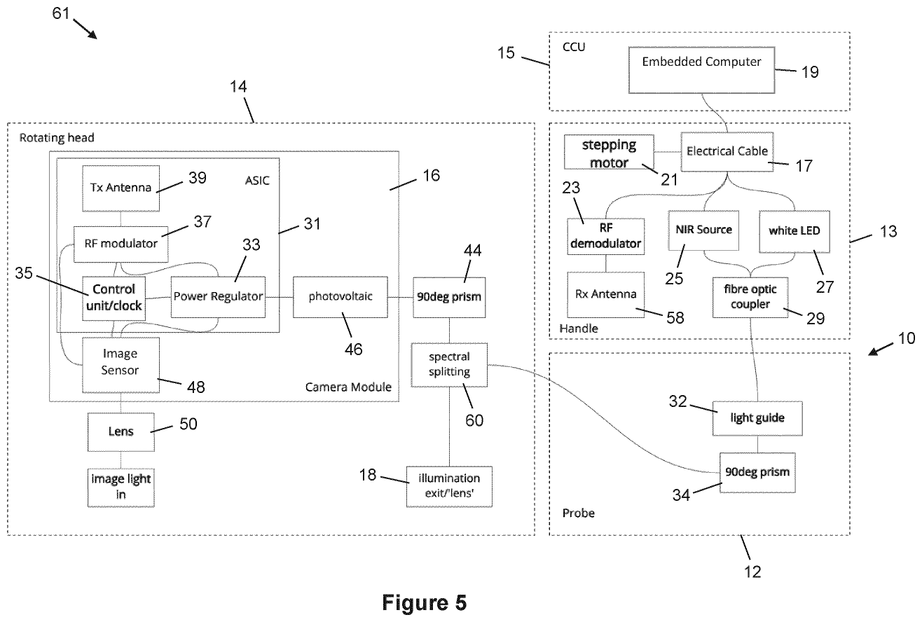

FIG. 5 is a block diagram of an endoscope system comprising the variant of FIG. 4;

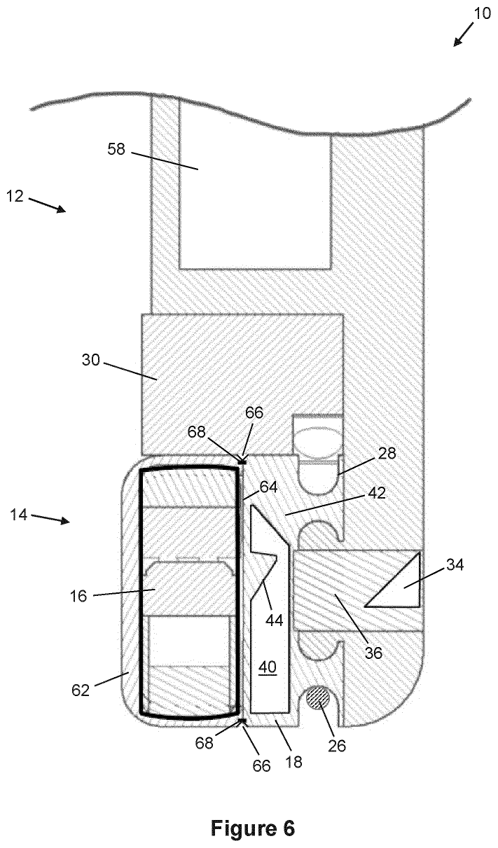

FIG. 6 corresponds to FIG. 2 and shows how an endoscope of the invention may be designed to facilitate remanufacture in a circular economy system;

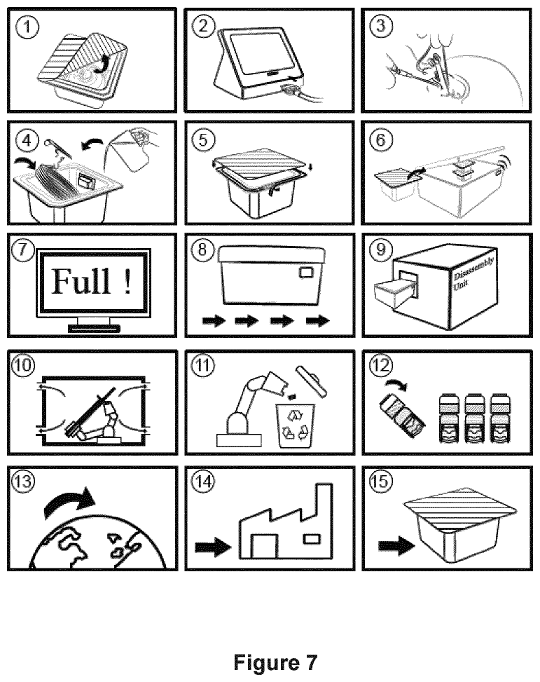

FIG. 7 is a sequence of schematic drawings that illustrate how the invention may be applied in a circular economy system;

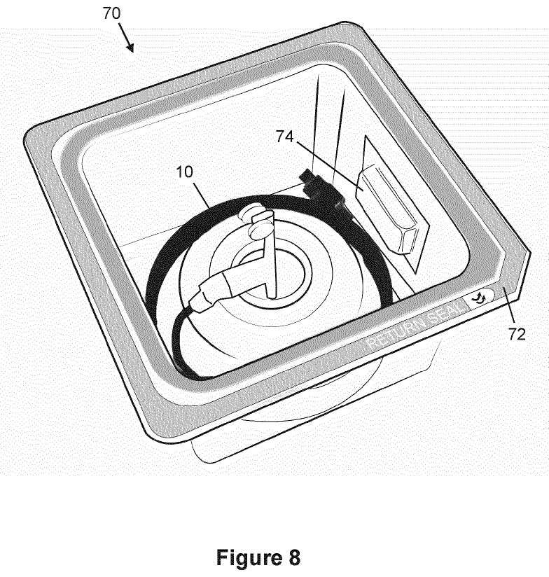

FIG. 8 is a perspective view of a tray for use in the method illustrated in FIG. 7;

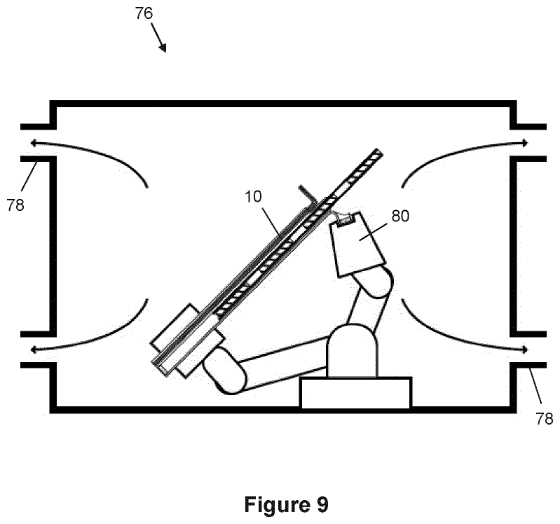

FIG. 9 is an enlarged detail view of a robotic cell used in one of the steps of the method illustrated in FIG. 7;

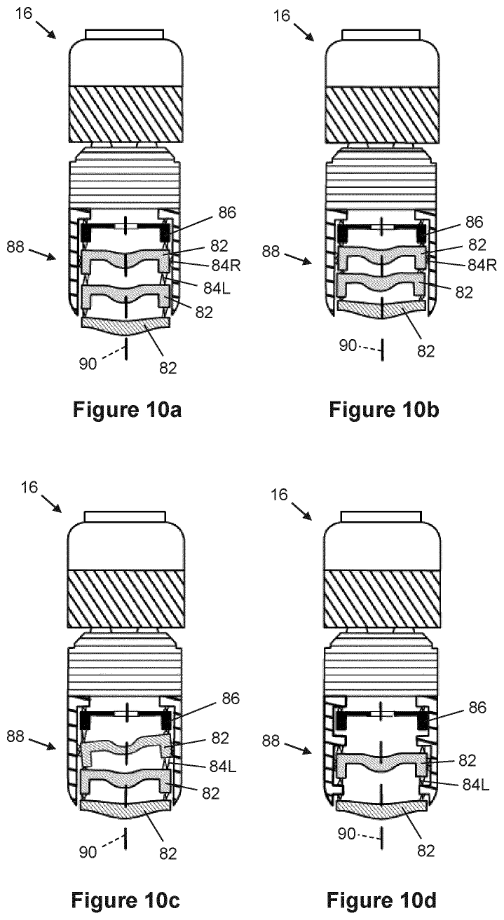

FIGS. 10a to 10d are side views in longitudinal section of camera module variants for use in endoscopes of the invention;

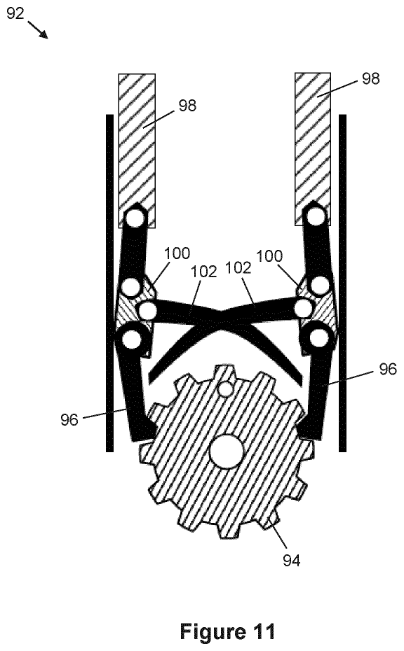

FIG. 11 is a schematic side view of a drive mechanism for rotating the head of an endoscope of the invention;

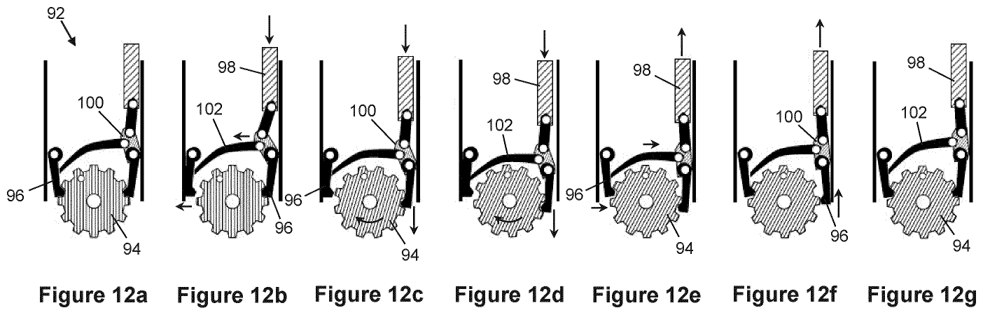

FIGS. 12a to 12g are simplified versions of FIG. 11, showing how the drive mechanism operates;

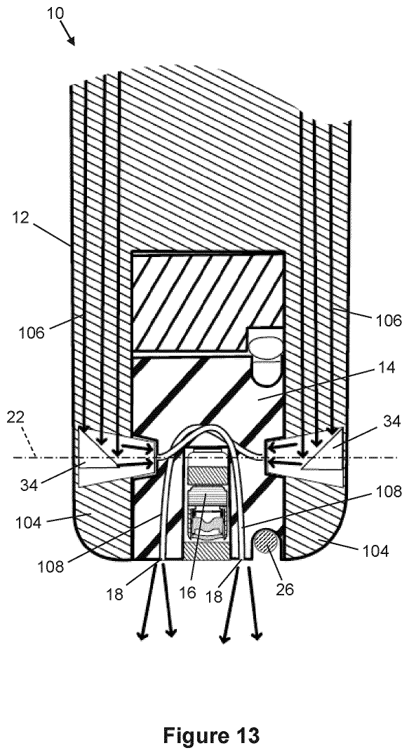

FIGS. 13 to 18 are side views of further embodiments of the invention, all in longitudinal section;

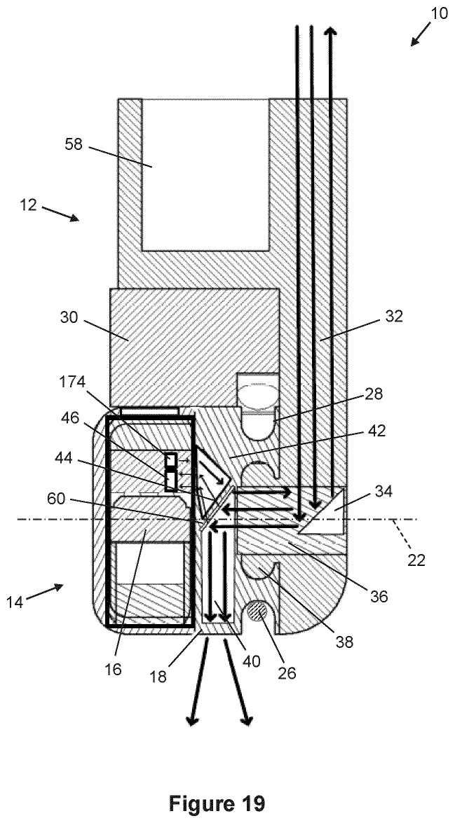

FIG. 19 is a side view in longitudinal section of a distal portion of a probe showing an optical pathway and components for transmission of data and power;

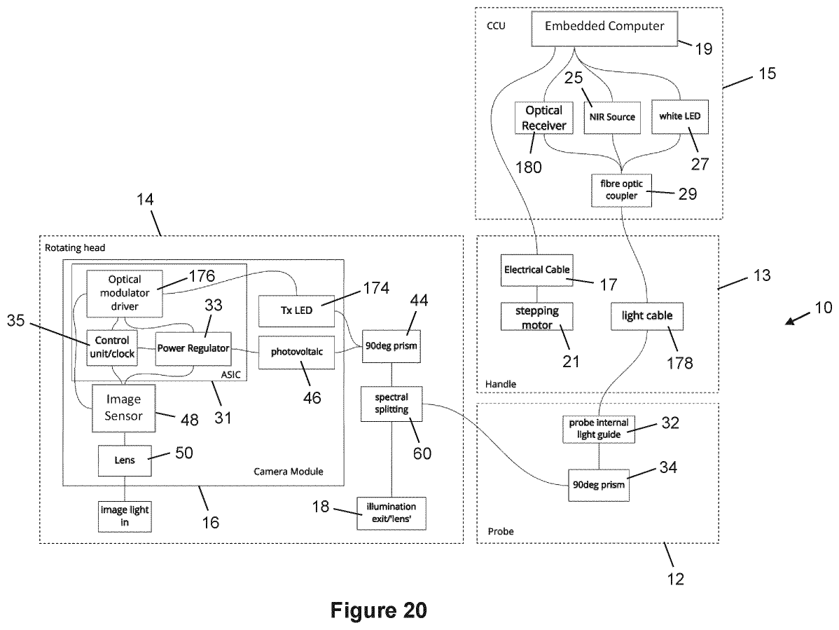

FIG. 20 is a block diagram of an endoscope system comprising the variant of FIG. 19;

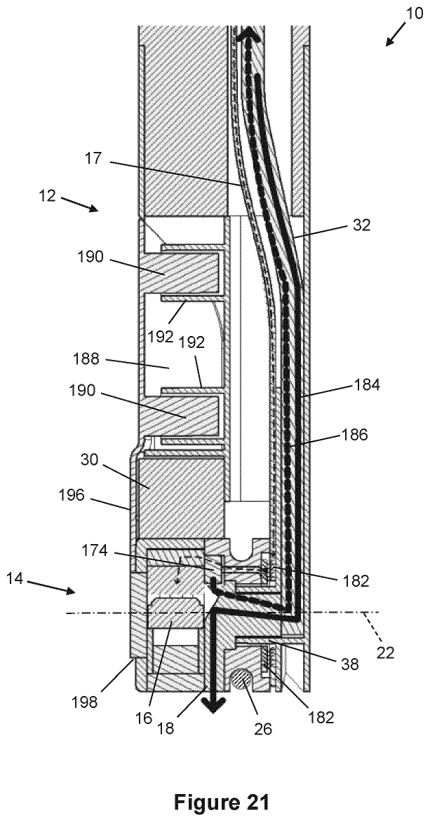

FIG. 21 is a side view in longitudinal section of a distal portion of a probe in which data is transferred optically and power is transferred wirelessly but non-optically;

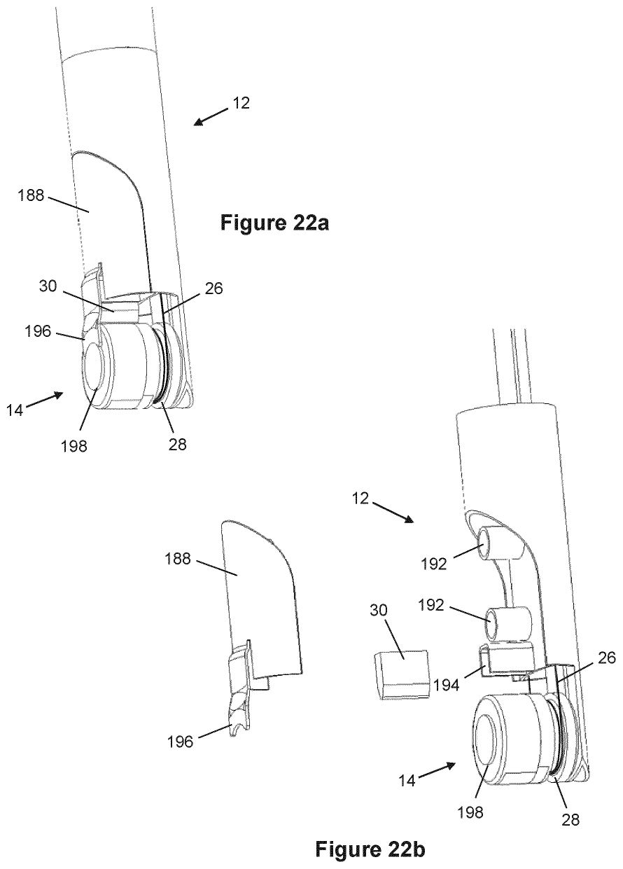

FIGS. 22a and 22b are detail perspective views of the probe of FIG. 21 in assembled and disassembled forms;

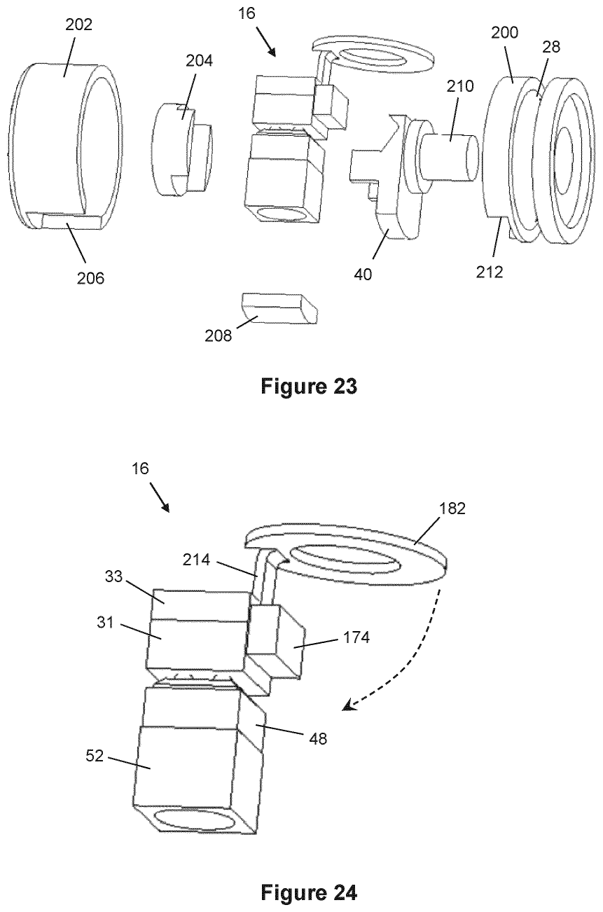

FIG. 23 is an exploded perspective view of a rotating head assembly for the probe of FIGS. 21, 22a and 22b;

FIG. 24 is an enlarged perspective view of a camera module of the rotating head assembly shown in FIG. 23;

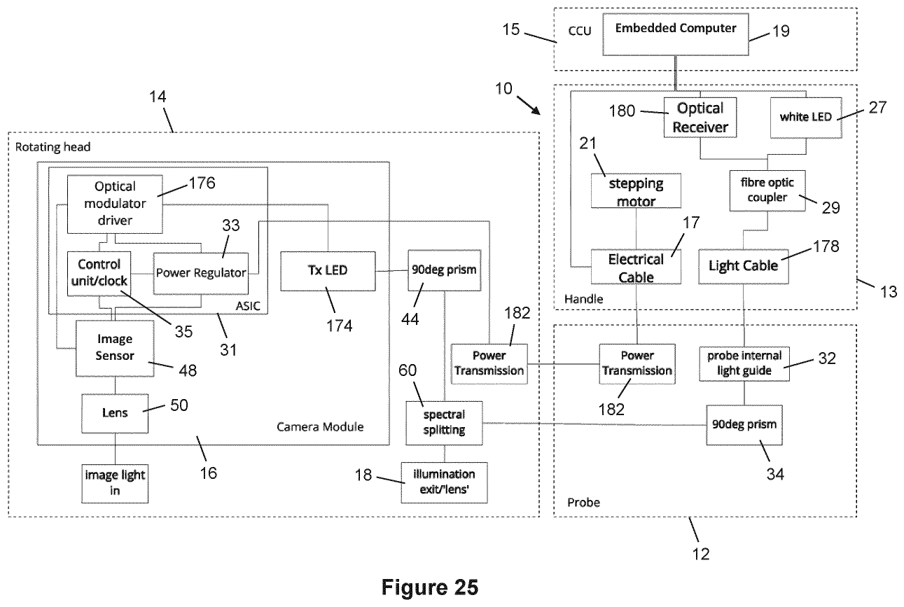

FIG. 25 is a block diagram of an endoscope system comprising the variant of FIG. 21;

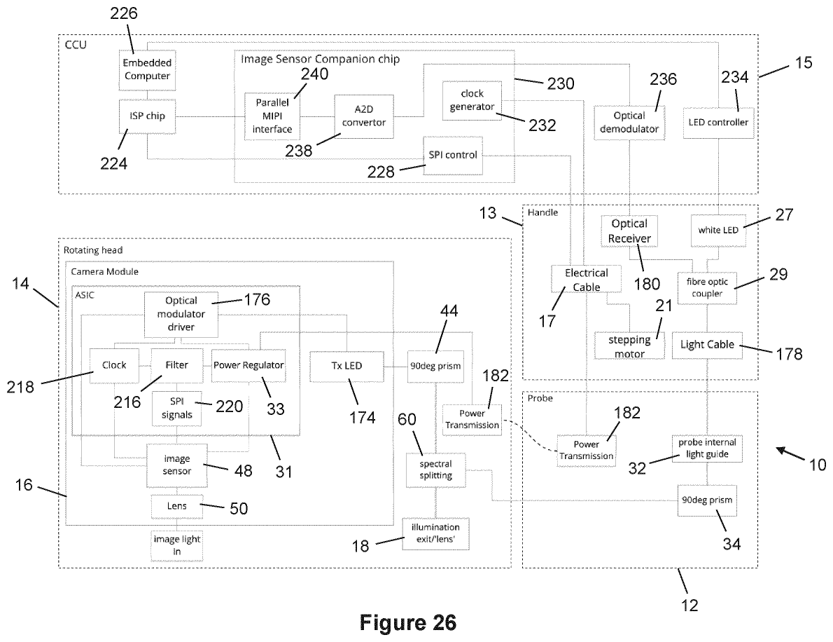

FIG. 26 is a block diagram of an endoscope system that addresses two-way data transmission;

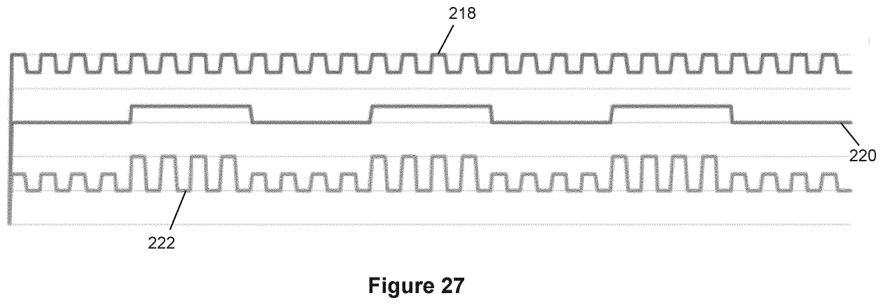

FIG. 27 is a diagram that illustrates data multiplexing;

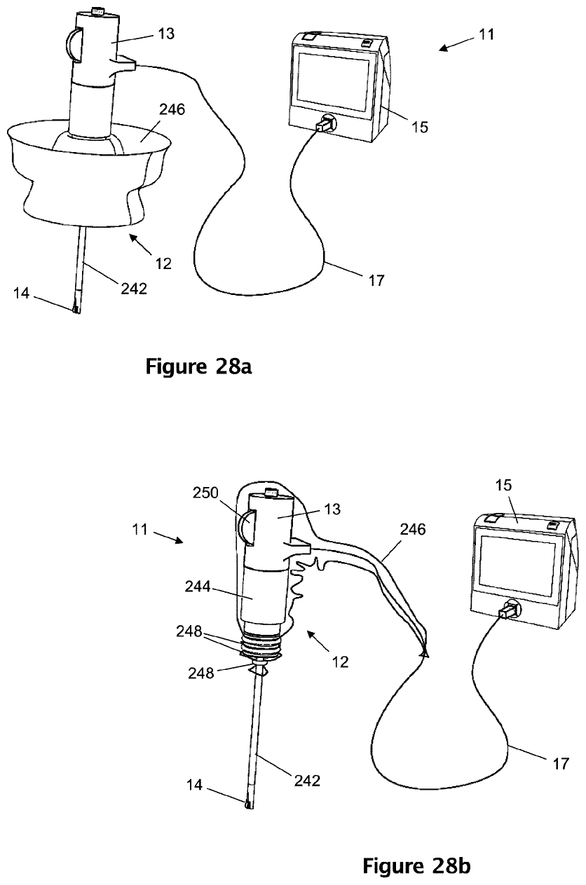

FIGS. 28a and 28b are perspective views of an endoscope system of the invention, comprising a probe assembly, a handle assembly and a camera control unit;

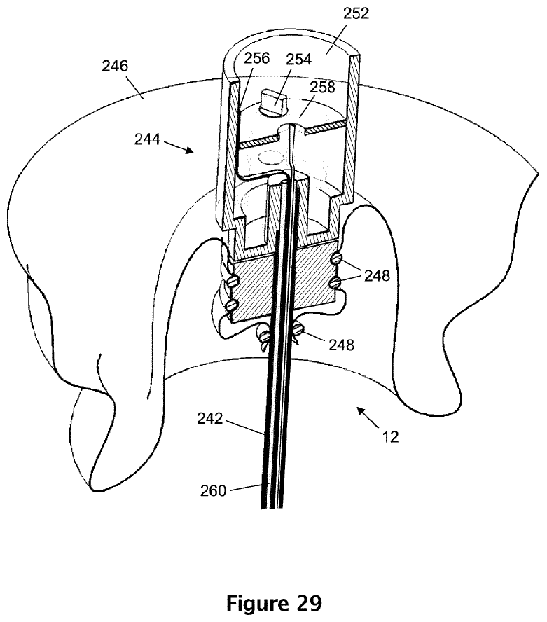

FIG. 29 is a sectioned perspective view of a proximal end of the probe assembly shown in FIGS. 28a and 28b;

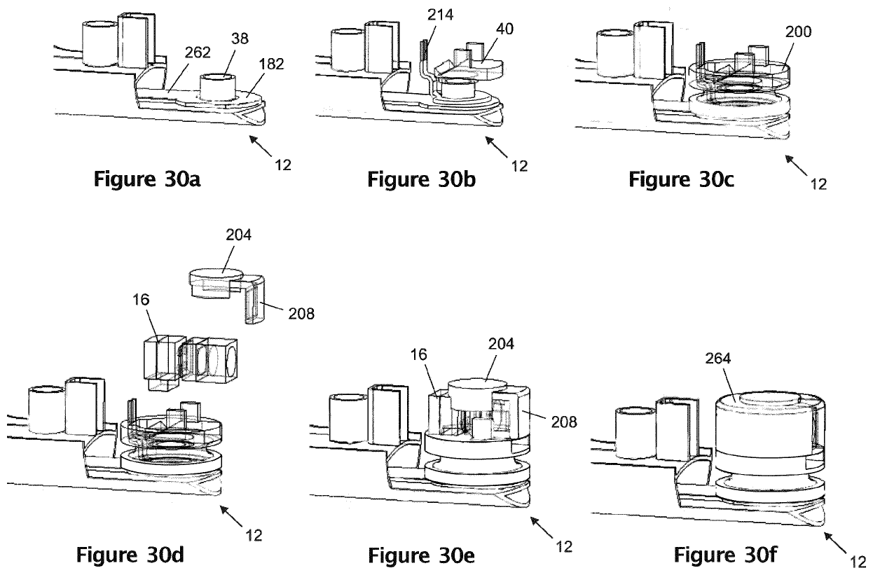

FIGS. 30a to 30f are a sequence of perspective views showing some of the steps involved in manufacturing the endoscope;

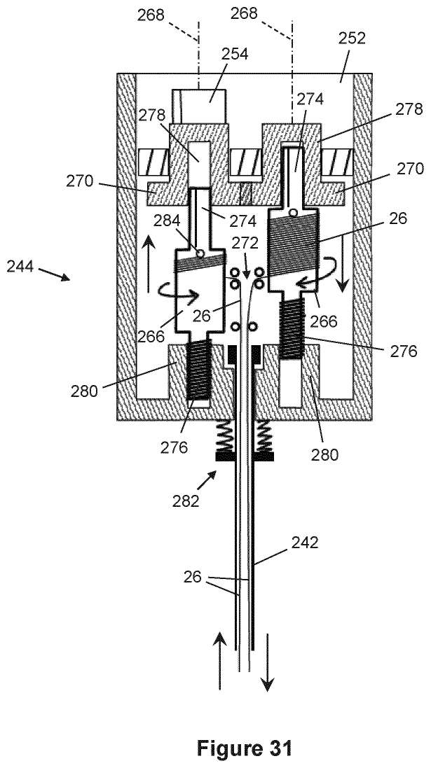

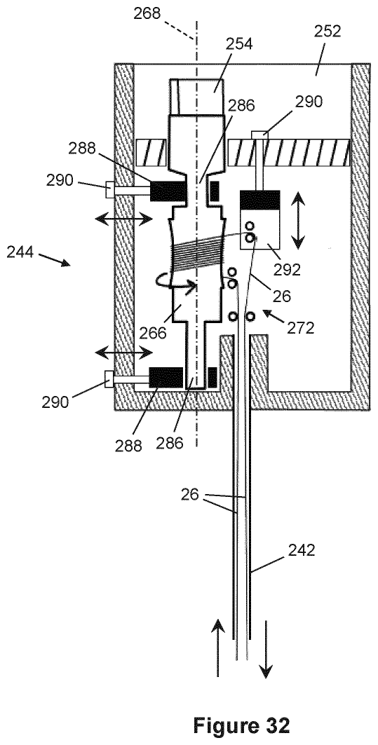

FIGS. 31 and 32 are schematic views in longitudinal section illustrating mechanisms for driving and managing the movement of a drive cable;

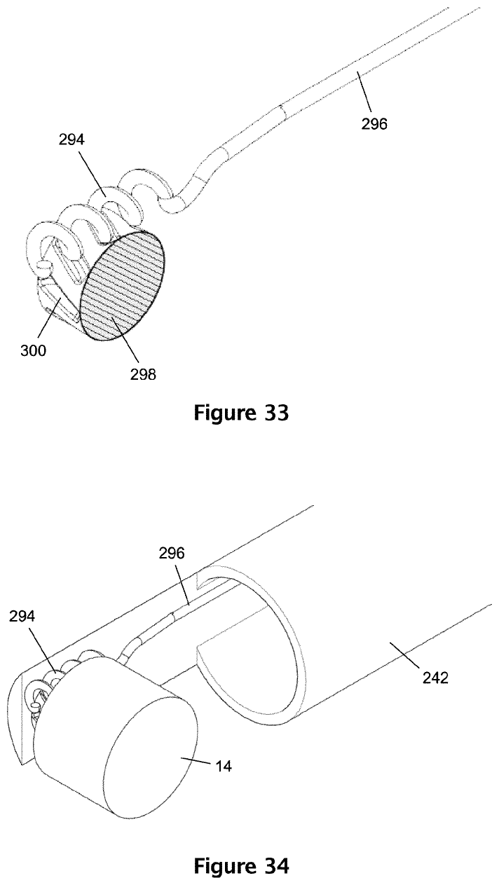

FIGS. 33 and 34 are perspective views illustrating a worm-gear mechanism for transmitting torque along a probe shaft to the head of the endoscope;

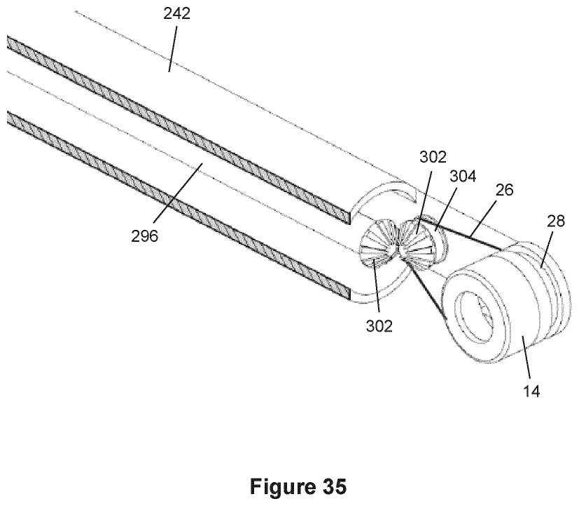

FIG. 35 is a perspective view illustrating a hybrid shaft- and belt-drive mechanism for transmitting torque along the probe shaft to the head of the endoscope;

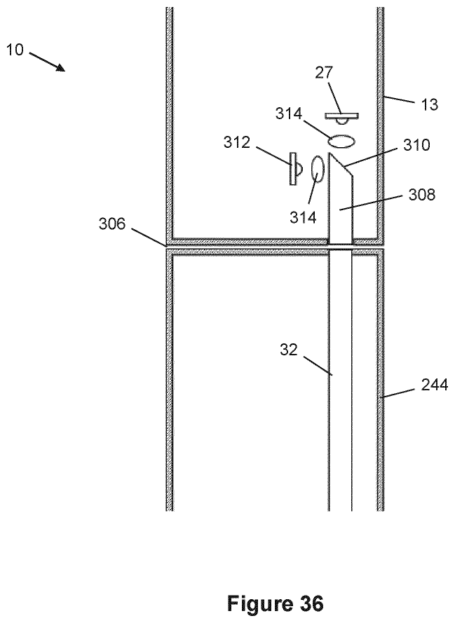

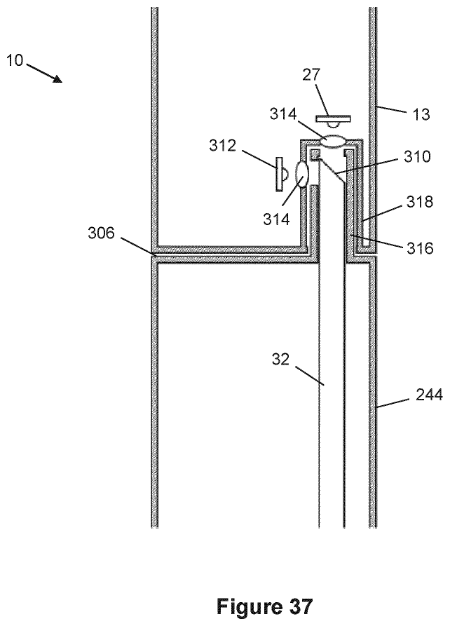

FIGS. 36 and 37 are schematic views in longitudinal section illustrating optical multiplexer arrangements;

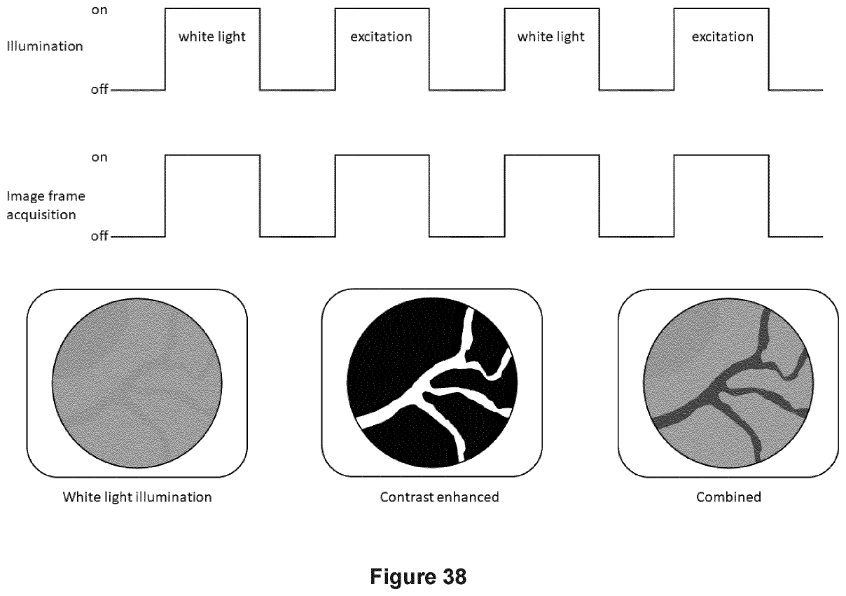

FIG. 38 illustrates visualisation of body structures during surgery;

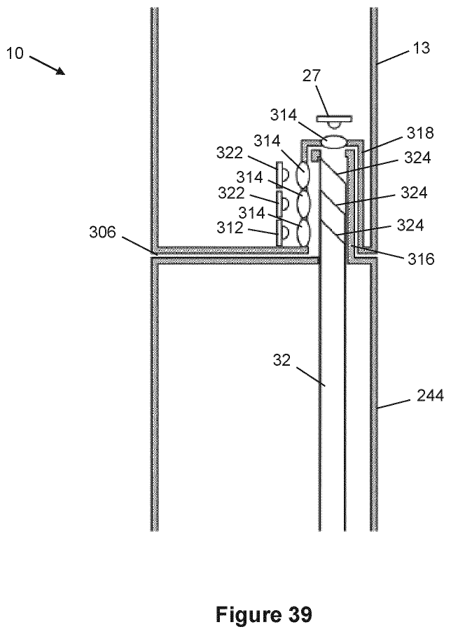

FIG. 39 is a schematic view in longitudinal section of an optical multiplexer arrangement suitable for use with the visualisation technique shown in FIG. 38;

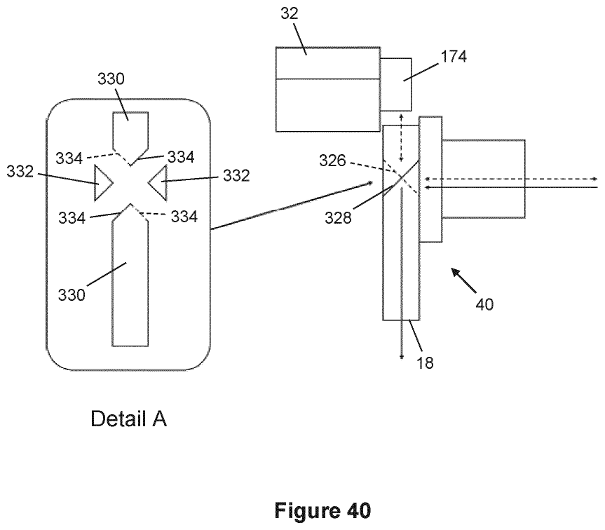

FIG. 40 is a schematic view of a light guide within the head of the endoscope with optical multiplexer/demultiplexer functionality;

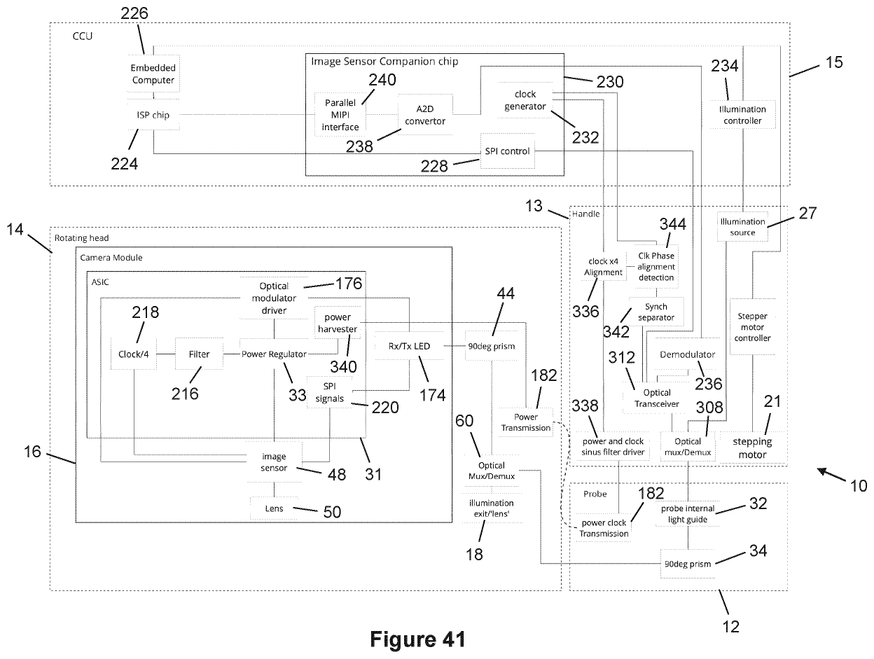

FIG. 41 is a block diagram of another endoscope system of the invention; and

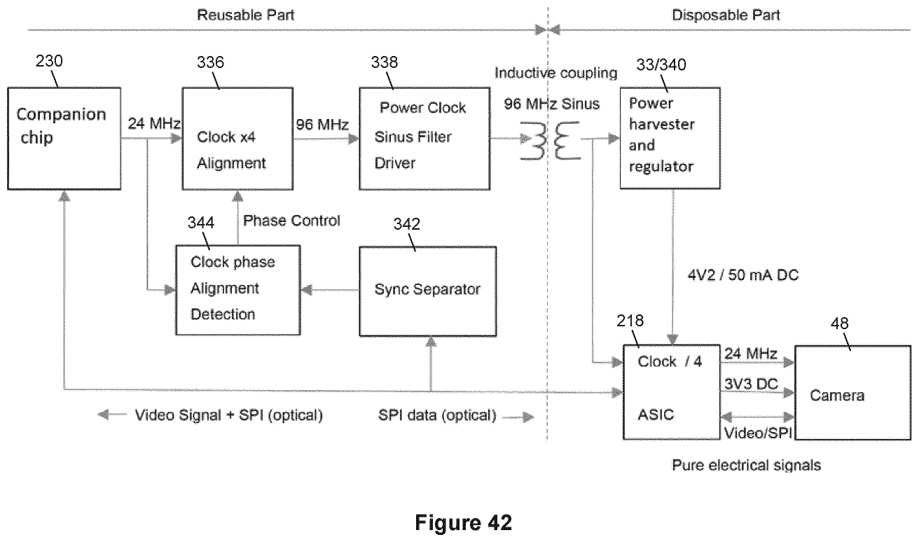

FIG. 42 is a detail block diagram of parts of the system shown in FIG. 41.

Referring firstly to FIGS. 1 to 3 of the drawings, a distal end portion of an endoscope 10 comprises a body in the form of an elongate stem, shaft or probe 12 that supports a rotating imaging head 14. The head 14 houses an integrated camera module 16 and a light emitter 18 disposed beside the camera module 16 to illuminate the field of view.

The probe 12 extends along a longitudinal axis 20. The head 14 turns relative to the probe 12 about an axis of rotation 22 that extends orthogonally with respect to the longitudinal axis 20. The camera module 16 has a field of view that extends radially with respect to the axis of rotation 22. Thus, the camera module 16 can be oriented through multiple angles of view by turning the probe 12 around its longitudinal axis 20 and by turning the head 14 around its axis of rotation 22. In FIG. 1, the camera module 16 and the adjacent light emitter 18 are shown at an angle of 45° to the longitudinal axis 20.

The head 14 is generally cylindrical, extending along and being rotationally symmetrical about the axis of rotation 22. The head 14 is supported for rotation by a mount in the form of a distally-extending cantilever arm 24 of the probe 12, offset laterally from the longitudinal axis 20. The cantilever arm 24 supports the head 14 at only one end or side of the head 14. This beneficially maximises the size of the head 14 within the constrained diameter of the endoscope 10, eases assembly and disassembly, provides greater freedom to arrange the internal components of the head 14 and leaves the free end or side of the head 14 available for additional lateral imaging if desired.

In this example, rotation of the head 14 is driven by a continuous drive cable 26 that extends from a drive mechanism (not shown) within the probe 12 and wraps around the head 14. The drive cable 26 is retained in a pulley groove 28 encircling the axis of rotation 22 adjacent to the end of the head 14 adjoining the cantilever arm 24. Rotation of the head 14 relative to the probe 12 about the axis of rotation 22 is unconstrained and so can exceed 360° in this example.

The probe 12 supports a distally-facing resilient wiper blade 30 that has an elongate free edge extending parallel to the axis of rotation 22. The wiper blade 30 bears against the proximal side of the head 14. As the head 14 is rotated past the wiper blade 30, the wiper blade 30 cleans debris from the head 14 to prevent that debris obscuring the field of view of the camera module 16 or blocking illumination from the light emitter 18.

Although not shown here, an outer lens of the camera module 16 may sit slightly proud of the surrounding outer surface of the head 14 so that the wiper blade 30 only impinges on the outer lens as the head 14 turns and does not drag on the remaining circumference of the head 14. Thus, there may be a small radial clearance between the wiper blade 30 and the head 14 except where the protruding outer lens of the camera module 16 bridges that gap on encountering the wiper blade 30.

In this embodiment, as will now be explained, the light emitter 18 is the distal end of an illumination conductor or light path that conveys light along the probe 12 to the head 14 from a remote light source. This solution is favoured in view of size constraints within the head 14 and because it allows easier variation of the light spectrum, for example by use of different light sources, which may be useful for tissue identification and in other respects.

Unlike transmission of electricity, transmission of light from the probe 12 into the head 14 does not require a complex contact or sealing arrangement. However, the problem of providing electrical power to the camera module 16 within the head 14 remains. In this embodiment, the solution is to generate the necessary electricity within the head 14 from a portion of the incoming light. In this way, a single efficient mechanism avoids the problem of sealing electrical contacts and better addresses the challenge of providing an illumination solution within a restricted space than the alternative of integral LEDs in the head. The mechanism shown also facilitates the abovementioned rotation and cleaning solutions.

The cross-sectional views of FIGS. 2 and 3 show internal features that define light transmission paths within the endoscope 10.

FIG. 2 is a view from a direction orthogonal to both the longitudinal axis 20 and the axis of rotation 22. This shows that light from an external light source (not shown) travels distally through an optical medium 32 in the probe 12, in a direction generally parallel to the longitudinal axis 20. On reaching the distal end portion of the probe 12, the light encounters a mirror or preferably a prism 34 mounted in the cantilever arm 24. This directs the light through an optical medium 36 in a direction generally parallel to the axis of rotation 22 to enter the head 14 via the sliding interface or gap at the junction between the cantilever arm 24 and the head 14. The optical medium 36 therefore serves as a light outlet from which light is conveyed or transmitted across the interface or gap to impinge on the head 14.

FIG. 2 also shows that the cantilever arm 24 comprises a spigot 38 that is engaged within a complementary socket of the head 14 to support the head 14 for rotation relative to the cantilever arm 24. The optical medium 36 extends within the spigot 38 along the axis of rotation 22 to the interface between the cantilever arm 24 and the head 14, thereby to emit light into the head 14 across that interface. In this example, the spigot 38 is substantially coplanar with the groove 28 in the exterior of the head 14 that accommodates the drive cable 26.

It will be apparent from FIG. 2 that the head 14 further comprises a light guide 40 behind the light emitter 18. Conveniently, the pulley component 42 defining the pulley groove 28 that receives the drive cable 26 and the socket that engages the spigot 38 of the cantilever arm 24 is integral with the light guide 40, for example as part of the same component or with internal features encapsulated within external structures.

The light guide 40 is disposed between the camera module 16 and the cantilever arm 24. In other words, the cantilever arm 24, the light guide 40 and the camera module 16 are disposed in succession along the axis of rotation 22. Thus, the light guide 40 defines part of the interface between the head 14 and the cantilever arm 24 and receives the light from the optical medium 36 within the spigot 38 of the cantilever arm 24. The light guide 40 therefore serves as a light inlet of the head 14 that receives light conveyed or transmitted from the light outlet of the cantilever arm 24 across the interface or gap at the junction between the cantilever arm 24 and the head 14.

On entering the head 14, the incoming light is split into two portions by the light guide 40. A first portion of the light impinges on a mirror or preferably a prism 44 of the light guide 40 and is thereby redirected radially away from the axis of rotation 22. That first portion of the light, thus redirected, is then conveyed through an optical medium of the light guide 40 to the light emitter 18, where it exits the head 14 to illuminate the field of view of the camera module 16.

A second portion of the light is directed through the light guide 40 to impinge on a photovoltaic cell 46 that thereby generates electrical power for the electronic components of the camera module 16. In this example, the photovoltaic cell 46 is conveniently integrated into the camera module 16 but in principle, the photovoltaic cell 46 could be separate from the camera module 16.

Reference is now also made to FIG. 3, which is a view from a direction orthogonal to the longitudinal axis 20 and along the axis of rotation 22, in longitudinal section through the camera module 16. This shows that the camera module 16 preferably has a stacked configuration. Specifically, the camera module 16 suitably comprises a commercially-available image sensor 48, onto which a lens barrel 50 and a lens 52 are mounted. The image sensor 48 has BGA (ball grid array) connections that are electrically connected to a power IC (integrated circuit) 54 to convey power to the image sensor 48 and to convey image data from the image sensor 48. The image sensor 48 could include image processing circuits, in which case fewer connections may be required to transmit image data to the power IC 54.

The power IC 54 preferably includes the photovoltaic cell 46 and power control circuitry to power the image sensor 48 and wireless data transmission circuitry implemented on a data IC 56. The power IC 54 also passes through data from the image sensor 48 to the data IC 56. The data IC 56 preferably includes an antenna and circuitry to transmit data in packets as required by wireless transmission protocols.

To minimise the power required for wireless data transmission, the data IC 56 of the camera module 16 conveniently transmits data from the image sensor 48 over a very short range, in this example to a wireless receiver 58 located in the probe 12 near the distal end of the probe 12.

Turning now to FIG. 4, this shows a variant of the embodiment shown in FIGS. 1 to 3 in which like numerals are used for like features. This variant differs by the addition of a filter or filter layer 60 applied to the prism 44 within the light guide 40 of the head 14. In this variant, the camera module 16 is powered by light that is tuned or filtered to a specific wavelength or spectrum, preferably outside the visible light spectrum. The filter layer 60 allows that specified light to pass through to the photovoltaic cell 46 of the camera module 16 while deflecting the remaining light to provide illumination via the light emitter 18. This allows independent control of the power transmitted to the camera module 16 and the visible light used for illumination of the target area, for example by varying the intensity or other parameter of one component of the supplied light relative to the other component of that light. More generally, filtering of the light can be done inside or outside the head 14 or the probe 12.

FIG. 5 is a block diagram of an endoscope system 11 comprising the endoscope 10 shown in FIG. 4, in which power is transferred optically from the probe 12 to the head 14 and data is transferred wirelessly from the head 14 to the probe 12 using RF. As also shown in FIGS. 27 (a) and 27 (b), the endoscope system 11 comprises a handle 13 that is removably attachable to the probe 12 and a camera control system 15 that is removably coupled to the handle 13 by an electrical cable 17 to power and control the handle 13 and the endoscope 10. For this purpose, the camera control system 15 comprises an embedded computer 19.

The handle 13 comprises a stepping motor 21 that activates a drive mechanism within the probe 12 to drive rotation of the head 14 about the axis of rotation 22 shown in FIG. 4. The handle 13 further comprises a wireless receiver in the form of a receiving antenna 58 that provides signals to an RF demodulator 23. The demodulator 23, in turn, returns signals to the embedded computer 19 of the camera control system 15 via the cable 17. The cable 17 also provides power to a near-infrared source 25 and to an LED 27 that emits white light. The outputs of the source 25 and the LED 27 are combined by a fibre optic coupler 29 and fed through the probe 12 via the aforementioned optical medium serving as a light guide 40 and then to the head 14 via the prism 34. In the head 14, spectral splitting is effected by the filter layer 60 whereby white light is directed to the light emitter 18 and near-infrared wavelengths are directed via the prism 44 to the photovoltaic cell 46 of the camera module 16.

In this example, the photovoltaic cell 46 of the camera module 16 powers an application-specific integrated circuit (ASIC) 31 that combines the functions of the aforementioned power IC 54 and data IC 56. The ASIC 31 comprises a power regulator 33, a control unit 35 with clock functionality, and an RF modulator 37 that receives signals from the image sensor 48 and that feeds signals to a transmitting antenna 39.

FIG. 6 shows how the embodiment of FIGS. 1 to 3 may have features to facilitate reuse of key components in a circular economy system. Again, like numerals are used for like features. Similar principles of construction may be applied to other embodiments, such as that shown in FIG. 4.

In view of the aforementioned problem of prion contamination, any structure defining an external surface or other surface of the endoscope 10 that could come into contact with organic material cannot be reused and must be removed from the device while maintaining the integrity of internal components that are not contaminated. For this purpose, the internal components are encapsulated. This ensures that once the endoscope 10 has been successfully dismantled and the encapsulation has been removed, the internal components that are to be reused cannot contaminate other components during reassembly, or in case of failure of the device and exposure of those components during surgery.

As shown in FIG. 6, the major active components of the head 14 including the integrated camera module 16 and the internal features of the light guide 40, such as the prism 44, are encapsulated to form a hermetic seal around those internal components. Specifically, the camera module 16 is encapsulated by a shroud 62 and as noted above, the light guide 40 is integrated with, or solidly encapsulated into, the component that also defines the pulley groove 28 and the spigot 38.

The shroud 62 interfaces with the light guide 40 at a planar joint 64 to assemble the head 14 as a single capsule. A seal between the light guide 40 and the shroud 62 is formed by over-moulding or optionally by bonding, welding or compression. The components within the light guide 40 and the shroud 62 are likewise over-moulded or optionally glued, welded or otherwise encapsulated using a compression fit.

A groove 66 surrounding the head 14 in alignment with the joint 64 creates a line of weakness at which the capsule can be broken to separate the light guide 40 and the shroud 62 in a controlled manner to remove the protected components inside. More generally, the groove 66 exemplifies a disassembly interface at which the camera module 16 is preferentially separable from other parts of the head 14 such as the light guide 40 and/or the light emitter 18. The groove 66 is external to the head 14 in this example but optionally, a line or point of weakness could be defined within the head 14.

Optionally, the groove 66 can be shaped to provide a hinge for controlled opening of the capsule, for example if the groove 66 does not extend around the entire circumference of the head 14.

FIG. 6 also shows an optional strip or band 68 of a harder material embedded within the head 14 under a cutting line, for example located under the groove 66 as shown. This allows a laser or knife to cut into the head 14 while protecting the internal components of the head 14 during the cutting process. For example, a laser could cut into a plastic capsule of the head 14 to open or weaken it, while being prevented from penetrating to the inside of the capsule by a metal band 68 located under the cut site.

Advantageously, the capsule of the head 14 may be opened by applying a bending or twisting force to its outer surface to tear, crack or break the capsule. Optionally, this action may be directed to a predetermined line or point of weakness as described above and/or may follow the application of a cut or scribe mark to the capsule as a preliminary step. Ideally, the required forces are applied to the outer surface of the capsule in a controlled and repeatable manner, for example by using a robotic arm.

Confirming that the capsule of the head 14 maintained its integrity during use is important to determine whether or not a component within that capsule can be used again. During disassembly, this check can be done visually, optionally by applying strain or colour dies or scanning frequencies to visualise cracks or to check resonance. Alternatively, or additionally, liquid or gas pressure could be applied to the outer surface of the capsule. If the capsule has failed, a void or pocket in the pressurised capsule would fill with fluid. Optionally testing can be in the form of a bubble leak test conducted with a pressure differential. This can be detected by visual monitoring or by monitoring pressure.

Manufacture of an endoscope 10 with encapsulated components will be described later with reference to FIGS. 30a to 30f of the drawings.

FIG. 7 exemplifies how the invention may be applied in a circular economy system. In this respect, it is important to protect the integrity of key components after use and to return them. Robust reverse logistics and supply chain management and focused product design and disassembly/reassembly processes are therefore required.

FIG. 7 shows an endoscope of the invention being removed from a sterile reusable tray (1), connected to a monitor or camera control unit (2) and used in ear surgery (3). After use, the endoscope is placed back in the original tray (4), although the tray in which the endoscope is supplied or returned could instead be single-use.

An example of a reusable tray 70 is shown in FIG. 8. In step (5) of FIG. 7, a user pulls a protective backing from a return seal 72 and then reseals the tray 70 around an endoscope 10, conveniently using the original covering film to close the open top of the tray 70. Advantageously, the user may firstly fill the tray 70 with water, thus activating a water-soluble detergent capsule 74 in the tray 70. The detergent initiates the cleaning process, loosening overt biological matter and debris from the endoscope 10.

Returning to FIG. 7, the tray 70 is then placed in a depository provided to individual hospitals (6). Depositories may provide geolocation and inventory data. When the depository is full, or nearly so, an alert is generated (7), leading to the depository being transported (8) to a specialised disassembly unit (9). This removes the burden of cleaning and waste management from hospitals and eliminates the risk of poor sterilisation.

A global product identification system allows individual serialisation and secure tracking of products and components and validation of the authenticity of those items as part of the returns process. This enables robust implementation of a smart Kanban (lean scheduling) system in which local distributors can be used to transport the endoscope to the disassembly unit.

Advantageously, disassembly units may be robotic cells of a size that makes shipping easy. A set number of such cells could fit into a standard shipping container, or a cell could be implemented in a shipping container. Such cells may be placed regionally or nationally as a key part of a reverse logistics system.

Thus, used endoscopes delivered directly from the hospitals, classified as medical waste, may be fed into robotic cells that serve as disassembly units. Initial disassembly of endoscopes is automated with a pick-and-place robot to protect staff. Robotic arms unpack and dismantle the endoscopes (10) and sort the parts into: parts for recycling (11); parts for reuse in the circular economy system (12); waste for incineration and biodegradable materials.

The parts for reuse in the circular economy system are no longer classified as medical waste and can be shipped internationally (13) to a circular production facility at which endoscopes are remanufactured (14). This solution avoids the many regulatory challenges that would have to be overcome if parts were shipped internationally while still classified as medical waste.

Each endoscope undergoes rigorous testing to ensure that it meets standards of safety and sterility equivalent to those of the original endoscope and is packaged ready for resale (15).

FIG. 9 shows the robotic cell 76 of a disassembly unit, as may be used in step (10) of FIG. 7. In this example, air is drawn from the cell 76 through ports 78 to apply a partial vacuum to the endoscope 10 during its disassembly by robotic arms 80. This is to discourage transfer of particles such as prions from the outer surface of the endoscope 10 to its internal protected components. The vacuum could be localised or transient or the entire disassembly process could take place in a low-pressure environment. Alternatively, laminar or other air flow may be applied to the endoscope 10 within the robotic cell 76 for the same purpose.

Moving on to FIGS. 10a to 10d, these drawings show variants of camera modules 16 that each have at least two lens elements 82, at least one of which is mounted on and movable by micro electro-mechanical system (MEMS) actuators 84. This enables the lens elements 82 to be moved independently of each other or in an accordion fashion. Optionally, the illustrated arrangements of MEMS actuators 84 could be replicated on all lens elements 82. MEMS actuators 84 could also act on a lens aperture 86 of the camera module 16 in a similar fashion.

A lens train 88 of three longitudinally-spaced lens elements 82 is shown in FIGS. 10a and 10b. Longitudinally-acting peripherally-positioned MEMS actuators 84L act on each lens element 82 to vary the longitudinal spacing between the lens elements 82. The effect of this is apparent from a comparison of FIG. 10a with FIG. 10b; the former drawing shows the lens train 88 expanded longitudinally whereas the latter drawing shows the lens train 88 contracted longitudinally. In this example, the lens elements 82 move relative to each other in accordion fashion and therefore the longitudinal positions of the lens elements 82 are inter-dependent.

FIGS. 10a and 10b also show that at least one lens element 82 can have further MEMS actuators 84R acting radially to keep that lens element 82, or more generally the lens train 88, in alignment. In principle, similar MEMS actuators 84R could also act on the lens aperture 86.

In FIGS. 10a and 10b, the lens elements 82 remain parallel and mutually aligned on a common longitudinal optical axis 90 as they move longitudinally. Conversely, the variant shown in FIG. 10c illustrates how MEMS actuators 84 on opposite sides of a lens element 82 could be controlled independently, for example by extending the MEMS actuators 84L on one side and retracting the MEMS actuators 84L on the other side. This causes at least one lens element 82 to tilt relative to the optical axis 90 and to at least one other lens element 82. Similarly, extending the MEMS actuators 84R on one side and retracting the MEMS actuators 84R on the other side will cause at least one lens element 82 to move transversely relative to the optical axis 90 and to at least one other lens element 82.

These pivoting and translational movements could be used to manoeuvre or manipulate a lens train 88 containing complex asymmetric lenses, allowing refocusing in a way that is not possible with simple lenses. For example, a lateral shift of a lens element 82 could bring a different optical shape onto the optical axis, thus refocusing the optical system.

In the variant shown in FIG. 10d, the lens train 88 comprises two lens elements 82. The lens elements 82 are suspended independently via MEMS actuators 84L and therefore are movable relative to a surrounding barrel structure independently of each other. The lens aperture 86 could also be mounted in a similar way. Again, provisions may be made for pivoting and/or translational movements like those described above.

Turning next to FIG. 11 and FIGS. 12a to 12g, these drawings show an orienting or drive mechanism 92 for turning the head 14, and hence the camera module 16 and the light emitter 18, relative to the probe 12 of the endoscope 10. The drive mechanism 92 is located near the distal end of the probe 12 and employs sliding fingers or pawls that could, for example, be powered using MEMS actuators.

Specifically, the drive mechanism 92 has a ratchet and pawl arrangement that comprises a ratchet wheel 94 coupled to the head 14, for example coaxially for direct drive or via the drive cable 26 of preceding embodiments. The drive mechanism 92 further comprises a pair of opposed pawls 96 that are movable as part of a chain or sequence of movements to engage with and apply torque to the ratchet wheel 94, thereby to drive and control angular stepwise movement of the ratchet wheel 94.

For this purpose, each pawl 96 is movable by a respective actuating rod 98 and linkage 100 to engage and disengage the ratchet wheel 94 and to apply torque to the ratchet wheel 94 when so engaged. A connecting arm 102 extending from each linkage 100 is driven by movement of that linkage 100 to act on the linkage 100 of the opposed pawl 96. In this way, extending one pawl 96 to advance the ratchet wheel 94 in a particular angular direction releases the other pawl 96 from the ratchet wheel 94 to free the rachet wheel 94 for that movement.

FIG. 11 shows the complete drive mechanism 92 required to turn the ratchet wheel 94 in opposite angular directions. For ease of understanding, however, FIGS. 12a to 12g omit one of the actuating rods 98, linkages 100 and arms 102 to illustrate unidirectional angular movement of the ratchet wheel 94. It will nevertheless be clear how these features omitted from FIGS. 12a to 12g but shown in FIG. 11 can be used to turn the ratchet wheel 94 in the opposite direction.

The sequence of operation shown in FIGS. 12a to 12g is as follows. Firstly, in the rest position shown in FIG. 12a and corresponding to FIG. 11, both pawls 96 are engaged with the ratchet wheel 94 to prevent angular movement of the head 14 relative to the probe 12.

FIG. 12b shows the actuating rod 98 of one of the pawls 96 driven longitudinally toward the ratchet wheel 94 along a tangential axis that is offset laterally from the centre of the ratchet wheel 94. Initially, this movement pivots the linkage 100 relative to the actuating rod 98 and the pawl 96. That pivotal movement of the linkage 100 moves the arm 102 attached to that linkage 100 toward and against the opposed pawl 96. This movement of the arm 102 disengages the opposed pawl 96 from the ratchet wheel 94 to free the ratchet wheel 94 for movement.

Clockwise movement of the ratchet wheel 94 is then driven by further longitudinal movement of the pawl 96 that is coupled to the actuating rod 98 via the linkage 100, as shown in FIGS. 12c and 12d.

Next, the actuating rod 98 reaches the end of its longitudinal stroke and starts to return in the opposite longitudinal direction as shown in FIG. 12e. Initially, this return movement pivots the linkage 100 in the opposite direction, pulling the arm 102 away from the opposed pawl 96. This allows the opposed pawl 96 to reengage the ratchet wheel 94, hence preventing angular movement of the ratchet wheel 94. FIG. 12f then shows the actuating rod 98 retracting further, hence disengaging the pawl 96 coupled to the linkage 100 from the ratchet wheel 94. Further retraction of the actuating rod 98 pulls that pawl 96 anticlockwise, sliding across at least one tooth of the ratchet wheel 94 to reengage the ratchet wheel 94 at a relatively anticlockwise position as shown in FIG. 12g.

The actuating rod 98 is now ready to repeat its longitudinal stroke if the ratchet wheel 94 is to be turned further in the clockwise direction. Alternatively, the actuating rod 98 can remain stationary if the ratchet wheel 94 is to be held at a fixed angular position or is to be driven in an anticlockwise direction by corresponding operation of the actuating rod 98, linkage 100 and arm 102 associated with the opposed pawl 96 as shown in FIG. 11.

FIG. 13 shows another embodiment of the invention in which like numerals are again used for like features. In this embodiment, the head 14 is not cantilevered from the probe 12 but is instead supported to turn about an axis of rotation 22 by, and between, a pair of laterally-spaced parallel arms 104 that extend distally from the probe 12.

Again, the head 14 contains and encapsulates a camera module 16 and supports at least one light emitter 18 that is positioned to illuminate the field of view of the camera module 16. In this example, there are at least two light emitters 18, one each side of the camera module 16.

Optionally, as shown in this example, light is introduced into the head 14 from parallel longitudinal light paths 106 extending along the probe 12. The light is redirected from those light paths 106 toward the head 14 by mirrors, light pipes or prisms 34 in the respective arms 104, aligned with the axis of rotation 22 in mutual opposition about the head 14. Thus, light enters the head 14 from opposite sides along the axis of rotation 22. From there, light guides 108 such as optical fibres within the head 104 convey the light to respective light emitters 18.

In this example, the camera module 16 could be electrically powered by conventional means such as sliding contacts or electromagnetic induction. However, it will be apparent that the camera module 16 shown in FIG. 13 could instead be powered by a photovoltaic arrangement using a portion of the light entering the head 14, for example using one or more light guides interposed between the camera module 16 and at least one of the arms 104 like the embodiment of FIGS. 1 to 3.

Again, features may be provided to facilitate robotic disassembly of the head 14 in a circular economy system, such as lines of weakness at which the head 14 can be cut or broken apart to access recyclable or reusable components encapsulated within.

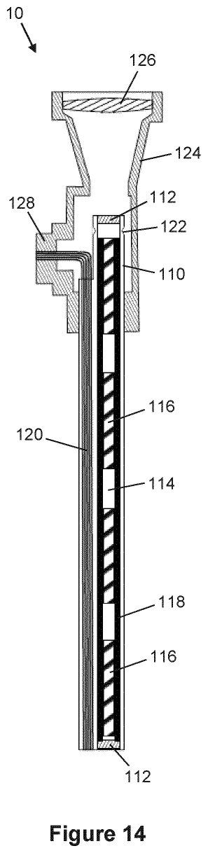

Moving on to FIGS. 14, 15 and 16, these drawings show embodiments of the invention in the form of endoscopes that resemble a traditional Hopkins® scope.

In the endoscope 10 of FIG. 14, a tubular capsule 110 has its ends closed with lenses or transparent end caps 112 to encapsulate a rod lens assembly 114. The rod lens assembly 114 comprises a longitudinal array of rod lenses 116 within a tubular housing 118, which may for example be of stainless steel. The metallic housing 118 of the rod lens assembly 114 lends stiffness to the longitudinal shaft of the endoscope 108, noting that the capsule 110 is a single-use component that may be moulded of polymer.

A lateral extension of the capsule 110 also houses and supports, but does not encapsulate, optical fibres 120 that extend parallel to the rod lens assembly 114. The optical fibres 120 terminate at the distal end of the endoscope 10 in a light emitter that is adjacent to the distal end cap 112 of the capsule 110. The distal end cap 112 serves as a distal lens of the endoscope 10.

The capsule 110 is encircled near its proximal end by a groove 122 defining a line of weakness for controlled disassembly, whereby the proximal end cap 112 can be removed or hinged away from the capsule 110. This allows the rod lens assembly 114 to be removed from the capsule 110 and reused. For example, the distal end cap 112 could be pressed proximally into the capsule 110 to push the rod lens assembly 114 proximally out of the capsule 110. That telescopic action could also break off or otherwise open the proximal end cap 112 if the proximal end cap 112 has not already been removed or opened. Preferably, the end caps 112 are spaced longitudinally from the rod lens assembly 114 to ensure that the rod lens assembly 114 is not damaged during removal from the capsule 110.

The capsule 110 is supported by a structural support 124 that surrounds and shrouds the proximal end of the capsule 110. The groove 122 is located within the support 124, which therefore protects the proximal end of the capsule 110 that is weakened by the groove 122. The capsule 110 must therefore be removed from the support 124 when access to the rod lens assembly 114 is required. The capsule 110 is then held so as to apply even force across the rod lens assembly 114 before a force is applied on the proximal side of the groove 122 to tear the capsule 110.

The support 124 also holds a proximal lens 126 in alignment with the rod lens assembly 114 and defines a connector 128 for coupling a light source to the optical fibres 120. The support 124 is preferably made from a biomaterial. Optionally, the optical fibres and the proximal lens could also be encapsulated in a similar manner to the rod lens assembly 114.

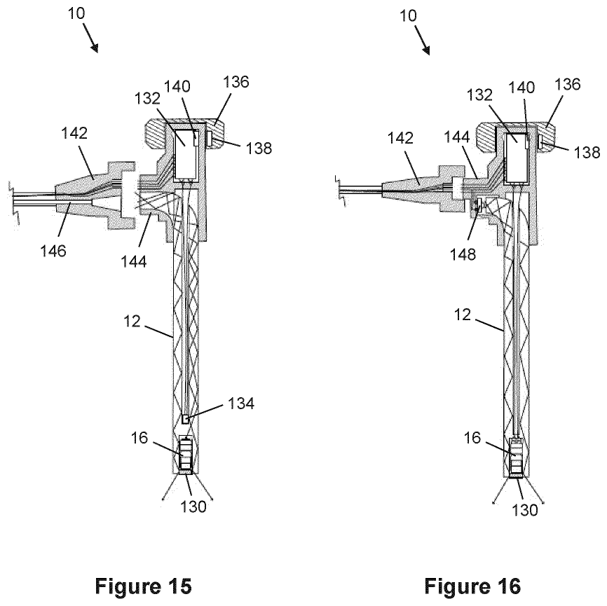

The endoscopes 10 shown in FIGS. 15 and 16 also resemble a traditional Hopkins® scope, with optical fibre light guide illumination, but in this case they each have an integrated, encapsulated camera module 16 at a distal end to provide a chip-on-tip solution.

Preferably, as shown, the camera module 16 includes a variable-focus lens that is controllable from the proximal end of the endoscope 10. For example, an objective lens 130 in front of the image sensor of the camera module 16 would allow focusing of the camera module 16 by varying the distance between them.

A separate encapsulated control module 132 near the proximal end of each endoscope 10 controls focus of the camera module 16 and receives image data from the camera module 16. In FIG. 15, the control module 132 receives data from an antenna 134 positioned close to the camera module 16 to receive that data wirelessly from the camera module 16. Conversely, the control module 132 has a wired data connection to the camera module 16 in FIG. 16.

Focus of the camera module 16 may conveniently be controlled by a proximal knob 136 that acts on the control module 132. In these examples, the knob 136 interacts with the control module 132 without physical contact between them. For this purpose, the knob 136 has reference markers 138 whereby a sensor 140 of the control module 132 can monitor the movement of the knob 136.

In the endoscopes 10 of FIGS. 15 and 16, a tubular shaft or probe 12 serves as a structural element and as a light guide. Light is conveyed along the probe 12 by internal reflection, as shown, to emerge from the distal tip of the probe 12 around the camera module 16. In the embodiment of FIG. 15, some of that light may power the camera module 16 via a photovoltaic cell. In the embodiment of FIG. 16, the camera module 16 may be powered directly by a wired connection to the control module 132.

Power and data connections to the control module are made via cables 142 that couple to connectors 144 of the endoscopes 10 in FIGS. 15 and 16. In FIG. 15, the cable 142 also includes fibre optics 146 to feed light from a remote light source, not shown, into the light guide of the probe 12 via the connector 144. Conversely, in FIG. 16, the cable 142 provides electrical power to an onboard light source within the endoscope 10, exemplified here by an LED 148 that feeds light into light guide of the probe 12.

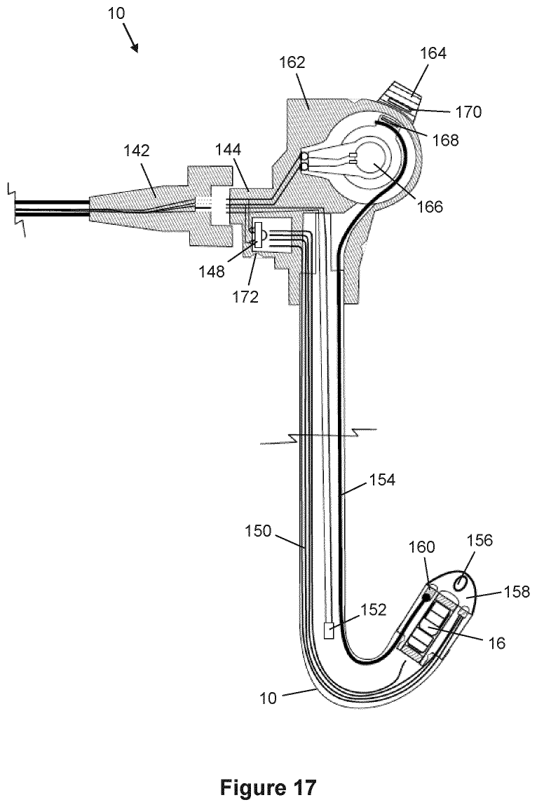

Turning next to FIG. 17, the endoscope 10 of this embodiment has a flexible tubular probe 12 that encloses optical fibres 150, a wireless communication module 152, and a steering wire 154 that is configured to bend the flexible probe 12 along its length. The probe 12 also accommodates a working channel that terminates in an exit opening 156 in a head component 158 at the distal tip of the probe 12.

In addition to defining the exit opening 158 for the working channel of the probe 12, the head component 158 encapsulates a camera module 16, directs illumination from the optical fibres 150 and provides a protective anchor for a plug 160 at the distal end of the steering wire 154. Some of the optical fibres 150 are directed to illuminate a photovoltaic cell that provides electrical power to the camera module 16. In conjunction with the probe 12 and a handle structure 162, the head component 158 also maintains protection of internal components of the endoscope 10 against ingress of contaminants.

The handle 162 includes a control element 164 to manoeuvre the steering wire 154 without direct physical interaction between the control element 164 and an internal mechanism 166 that is encapsulated within the handle 162. For this purpose, a sensor 168 of the mechanism 166 monitors movement of a reference marker 170 on the control element 164, causing the mechanism 166 to respond to such movement by pushing or pulling the steering wire 154 to the desired extent. Optionally, the mechanism 166 is assisted or powered by a motor. Points of weakness 172 may be provided for controlled disassembly of the handle 162 to extract the encapsulated mechanism 166 for reuse.

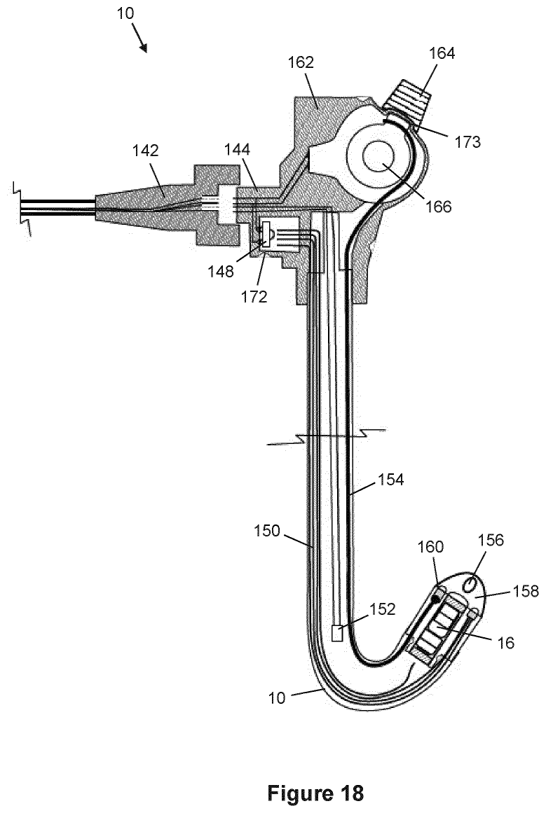

In the variant of FIG. 18, the sensor 168 and reference marker 170 of FIG. 17 are omitted. Instead, the control element 164 interacts with the internal mechanism 166 through a flexible membrane 173 that provides a sterile barrier between the internal mechanism 166 and the exterior of the endoscope 10.

Moving on now to FIGS. 19 to 27, these drawings show various other wireless arrangements for powering the components of the head 14 and for receiving data from the head 14.

In the variant shown in longitudinal section in FIG. 19 and as a block diagram in FIG. 20, power and data are transferred to and from the head 14 optically. For this purpose, a data-transmitting LED 174 is shown in FIG. 18 emitting data signals as light to the prism 44 in the head 14, which diverts that light to the prism 34 in the probe 12 and from there into the light guide 32.

The layout and components shown in the block diagram of FIG. 20 are similar to those of the block diagram of FIG. 5, and like numerals are used for like features. For brevity, the following description will focus on the main differences relative to FIG. 5.

In the camera module 16 of FIG. 20, the RF modulator 37 and the transmitting antenna 39 of FIG. 5 are replaced by an optical modulator driver 176 in the ASIC 31 that feeds data signals to the transmitting LED 174.

In this example, the near-infrared source 25, the white LED 27 and the fibre optic coupler 29 are implemented in the camera control unit 15. Here, therefore, the coupler 29 is coupled to the light guide 32 in the probe 12 via a light cable 178 extending through the handle 13 to the probe 12. The light cable 178 not only conveys light toward the head 14 but also receives light from the LED 174 of the head 14 via the light guide 32, for example via the coupler 29 as shown. The light cable 41 conveys the received light to an optical receiver 180 in the camera control unit 15. The wavelength used for optical data transmission from the LED 174 is distinct from the wavelengths of white light transmission so that it can be distinguished and detected by the optical receiver 180. The optical receiver 180 feeds the resulting signals to the embedded computer 19 in the camera control unit 15.

Whilst the optical receiver 180, the near-infrared source 25, the white LED 27 and the fibre optic coupler 29 are implemented in the camera control unit 15 in this example, any or all of them could instead be implemented elsewhere outside the head 14, such as in the handle 13 or in the probe 14.

In the variants shown in FIGS. 21 to 27, data is transferred optically from the head 14 as in the preceding embodiment whereas power is transferred wirelessly to the head 14 by non-optical means such as by RF or by near-field inductive coupling. The latter possibility is illustrated in FIG. 21, where power is transmitted inductively between mutually-confronting coils 182 that encircle the spigot 38 of the probe 12 relative to which the head 14 turns. One coil 182 is on the probe 12 and the opposed coil 182 is on the head 14.

The coil 182 of the probe 12 is connected to an electrical power cable 17 extending along the probe 12 in parallel relation to the light guide 32. The light guide 32 conveys illuminating light to the light emitter 18 of the head 14 on an outbound path 184. The light guide 32 also conveys light carrying a data signal from the data-transmitting LED 174 of the head 14 on an inbound path 186.

FIGS. 21, 22a and 22b also show a removable cover insert 188 that is held on the probe 12 by engagement of spigots 190 on the insert 188 within complementary sockets 192 on the probe 12. The insert 188 retains a cleaning module such as the wiper blade 30 in a receptable 194 that is integral with the probe 12. The insert 188 also has a concave hub bearing formation 196 that engages a complementary convex shoulder formation 198 centred on the axis of rotation 22 on an outer side of the head 14, opposed to the spigot 38. Thus, the insert 188 provides for assembly of the wiper blade 30 onto the probe 12 and for removal of the head 14 from the probe 12. The insert 188 also supports the wiper blade 30 and the head 12 in the assembled probe 12. Conversely, the insert 188 facilitates disassembly for circular manufacturing.

FIGS. 22a and 22b show how the drive cable 26 can be wrapped around the head 14 within the pulley groove 28. In this example, the drive cable 26 turns through about 540°, comprising one full circumferential loop of 360° around the head 14, plus an approximately half-circumferential turn, hence of nominally 180°, between the points where tangential legs of the drive cable 26 extend proximally from the pulley groove 28 into the probe 12. Thus, the drive cable 26 wraps around the head 14 once by a 180° turn and then twice by a 360° turn.

In another example, there could be no full loop of the drive cable 26 around the head 14, in which case the drive cable 26 would simply wrap or bend once around the head 14 by an approximately half-circumferential turn of about 180°. In other examples, the drive cable 26 could encircle the head 14 two or more times in the manner of a capstan. For example, there could be two full loops of 360° each or 720° on aggregate, meaning that the drive cable 26 turns through about 900° in total. Again, this includes an approximately half-circumferential turn of about 180° to allow for convergence of the drive cable 26 with, and divergence of the drive cable 26 from, the pulley groove 28.

FIG. 23 shows an exemplary assembly of components of the head 14. In this example, a pulley housing 200 and a camera housing 202 enclose and sandwich the camera module 16 and the light guide 40 in addition to an axis structure 204 that cooperates with the light guide 40 to locate the camera module 16 in the camera housing 202. The assembly further comprises a slot 206 in the camera housing 202 that receives a window 208 for admitting light to the camera module 16.

In a preferred sequence, the camera module 16 is assembled first with the window 208, the light guide 40 and the axis structure 204, which are preferably each designed to grip or locate the camera module 16 between them. The pulley housing 200 is then added to the assembly, followed by the camera housing 202. The pulley housing 200 is placed around a cylindrical projection 210 of the light guide 40 and the light guide 40 engages in a slot 212 in the pulley housing 200. This engagement locks the pulley housing 200 against angular movement relative to the remainder of the assembly.

The pulley housing 200 and the camera housing 202 could instead be formed over the other components of the assembly by an overmoulding process or by low-pressure injection moulding of adhesive, as will be described with reference to FIGS. 30a to 30f.

The camera module 16 is illustrated in more detail in FIG. 24, showing the lens 52, the image sensor 48, the ASIC 31 and its power regulator 33, the data-transmitting LED 174 and a power flex cable 214 comprising the coil 182 of the head 14. The power flex cable 214 is shown in FIGS. 23 and 24 in an initial position in which it is bent up to place the coil 182 at 90° to its final position after assembly. In that final position, the power flex cable 214 is bent down, as shown by the dashed arrow in FIG. 24, such that the coil 182 lies over the cylindrical projection 210 of the light guide 40, parallel to the plane of the circumferential groove 28 in the pulley housing 200.

The layout and components shown in the block diagram of FIG. 25 are similar to those of the block diagram of FIG. 20, and like numerals are used for like features. The main difference relative to FIG. 20 is the provision of the coils 182 that effect power transmission from the probe 12 to the head 14. The coil 182 of the probe 12 is shown here connected to the power cable 17 that also powers the stepping motor 21. Conversely, the coil 182 of the head 14 is shown connected to the power regulator 33 of the ASIC 31.

As the only light provided to the head 14 is for the purpose of illumination and as power is now supplied to the head 14 by other means, the near-infrared source 25 of FIG. 20 is omitted. However, the light emitted from the data-transmitting LED 174 is still subject to spectral splitting or filtering at 60. This is to reduce reflection of light from the light emitter 18 back along the light guide 32, which could otherwise interfere with the ability of the optical receiver 180 to differentiate between the reflected light and the data signal from the LED 174.

Another difference of the block diagram of FIG. 25 relative to FIG. 20 is that the optical receiver 180, the LED 27 and the fibre optic coupler 29 are returned to the handle 13 from the camera control unit 15.

Further to help the optical receiver 180 to differentiate the data signal from light that could be reflected back along the light guide 32 from the probe 12 or the head 14, illumination of the LED 27 that emits white light may be synchronised with the frame rate of the image sensor 48. In particular, illumination of the LED 27 may be on only when the image sensor 48 takes a frame image and hence off in the intervals between frames. The interval periods in which illumination of the LED 27 is off can be used to transmit optical data without an overlap in signal, thus making signal processing easier. A similar provision can be made not only in the arrangement shown in FIGS. 21 to 25 but in any arrangement in which data is transmitted optically, including that of FIGS. 19 and 20.

More generally, techniques used in modern electronics dictate that one-way data transmission from the image sensor 48 to the camera control unit 15 is not always an option and an element of two-way data communication may therefore be required. In this respect, FIG. 26 develops the arrangement of FIG. 25 to address two-way data transmission. Here, data is transmitted back to the camera control unit 15 using the light guide 32 as before but the wireless power transmission circuitry has a dual purpose of not only transmitting power but also transmitting data to the image sensor 48. That data 48 is in the form of a clock signal and a serial peripheral interface (SPI) control signal, namely an SPI write signal. The ASIC 31 of the camera module 16 comprises a filter 216 that differentiates between the clock signal at 218 and the SPI control signal at 220.

As the power transmission circuitry only provides for one channel, signal multiplexing is used. Specifically, to transmit a clock signal and SPI write data, the channel must transmit at least three different signal amplitude levels such as 0%, 50% and 100%. The clock signal is represented by 0% amplitude for the low portion of that signal and 50% amplitude for the high portion of that signal. For the short period of an SPI write data transfer, the 100% amplitude level is used to encode a ‘high clock with high SPI write data signal’, whereas the 50% amplitude level encodes a ‘high clock with low SPI write data signal’. This is equivalent to amplitude modulation of the clock signal by the SPI write data signal.

The multiplexing principle is illustrated in FIG. 27, where a clock signal 218 and an SPI control signal 220 are combined into a multiplexed signal 222. As a bit of the SPI write data signal lasts for four clock cycles, modulation is simple.

An inductive coupling is proposed for power transmission, in which a pair of coupling coils 182 are used to transfer power electromagnetically along with the clock signal 218 and/or other signals. Power may be harvested by an energy harvester chip and then regulated using a power regulator to drive a camera module 16 on the disposable side of the endoscope 10. Double-sided flexible PCB planar coils 218 can be used for transmission and reception of the power signal. The number of turns in the coils 218 may, for example, be from four to eight turns per side. A receiver coil 182 can also have one or more windings on the cylindrical projection 210 of the light guide 40 to improve inductive reception of power and associated signals.

Returning to FIG. 26, this drawing further elaborates on the functionality of the camera control unit 15 in this context. An image signal processing chip 224 responsive to an embedded computer 226 drives an SPI controller 228 implemented on an image sensor companion chip 230. The SPI controller 228 feeds SPI write data to the electrical cable 17 that powers the coil 182 of the probe 14. The image sensor companion chip 230 also implements a clock 232 whose signal is also fed to the cable 17, multiplexed with the SPI write data generated by the SPI controller 228.

In a further variation, the LED 174 can act in two directions, both transmitting and receiving optical data. This allows the SPI control signal 220 to be transmitted optically to the rotating head.

The computer 226 also drives an LED controller 234 that, in turn, controls the LED 27 to generate illuminating white light. Reciprocally, an optical demodulator 236 receives and demodulates signals from the optical receiver 180 and feeds its output to the image signal processing chip 224 via an analog-to-digital converter 238 and a parallel mobile industry processor interface 240.

Turning next to FIGS. 28a and 28b, these exemplify an endoscope system 11 of the invention comprising three main components, namely a probe assembly 12 including a rotating head 14 at a distal end of a shaft 242; a handle assembly 13; and a camera control unit 15 connected to the handle assembly 13 by a cable 17. In this example, the handle assembly 13 and the camera control unit 15 are reusable whereas the probe assembly 12 is single-use or subject to circular manufacturing so as to be provided in a sterile state for each surgical procedure.

The probe assembly 12 has a cylindrical body 244 that supports and is surrounded by an over-sheath 246. The over-sheath 246 is secured to the body 244 by a series of O-rings 248 that encircle both the over-sheath 246 and the body 244. The over-sheath 246 can be pulled over the handle assembly 13 as shown in FIG. 28b to provide a sterile barrier around the handle assembly 13.

The handle assembly 13 includes cables and connectors necessary to interface with the probe assembly 12 and the camera control unit 15. Optionally, the handle assembly 13 includes controls 250 for adjusting the head 14 and for initiating cleaning or wiping of the camera module 16. The handle assembly 13 may also include the aforementioned stepping motor 21 acting on a mechanism to rotate the head 14, along with electrical contacts, an optical receiver 180, LEDs and PCBs.

The probe assembly 12 includes conduits for conveying power, data, illumination and mechanical movement to and/or from the rotating head 14. With reference to the proximal end view of the probe assembly 12 of FIG. 29, this shows features that interface with the handle assembly 13, namely a socket 252 for receiving a distal end of the handle assembly, a mechanical rotation interface 254 or dog clutch for receiving drive from the stepping motor 21, electrical contacts 256 and an optical interface 258. FIG. 29 also shows that the shaft 242 of the probe 12 is of multi-lumen construction and is supported by an internal structural wire 260.

Turning next to FIGS. 30a to 30f, these drawings illustrate ways of meeting the challenges of manufacturing an endoscope of the invention. In this respect, the extreme miniaturisation of the head 14, in particular, means that novel approaches are beneficial during manufacturing and assembly.

FIGS. 30a to 30f show a sequence of steps that deploy overmoulding techniques in a clean room assembly line for building a subassembly of an endoscope 10.

Overmoulding may be done using micro injection moulding or low-pressure adhesive injection moulding techniques.

FIG. 30a shows a distal structure of the probe 12 with a power flex cable 262 in place, a coil 182 of the power flex cable 262 encircling a spigot 38 that defines the axis of rotation 22. This assembly is positioned in a mould for overmoulding. Next, FIG. 30b shows a power flex cable 214 of the camera module 16 and the light guide component 40 as they too would be assembled into the mould.

FIG. 30c shows the pulley housing 200 now overmoulded on top of the abovementioned previously-placed components. Next, with reference to FIG. 30d, the resulting assembly is placed in a second mould for overmoulding again, before which the camera module 16, axis structure 204 and window 208 components are positioned. This step also allows for an electrical connection to be made between the camera module 16 and the power flex cable 214 of the camera module 16.

FIG. 30e shows the assembly ready for the second overmoulding process and FIG. 30f shows the assembly with the resultant camera housing overmould 264 in place. This defines the head 14 assembled onto the distal structure of the probe 12, with the head 14 being rotatable on the spigot 38 of the distal structure. The tightness of the assembly prevents ingress of fluids past the spigot 38 and the pulley housing 200 interface.

Importantly, the camera module 16 that houses the most expensive components, such as electronic components comprising rare metals, is completely encapsulated in this process. This is advantageous as it protects the camera module 16 from contamination with prions or other contaminants that are difficult or impossible to remove, hence allowing for disassembly and reuse of the camera module 16 in a circular manufacturing process.

Moving on to FIGS. 31 and 32, these drawings show optional mechanisms for driving and managing the movement of a drive wire, belt or cable 26. With reference to FIGS. 28b and 29, the mechanisms shown in FIGS. 31 and 32 are accommodated within a cylindrical body 244 at the proximal end of the shaft 242 of the probe assembly 12. Portions of the drive cable 26 extend parallel to each other along the shaft 242 to turn the head 14 of the probe at the distal end of the shaft 242.

As before, a proximal socket 252 of the body 244 receives and interfaces with a distal end formation of the handle assembly 13, which is not shown in these drawings. In each case, the mechanisms are powered by a drive arrangement comprising the aforementioned stepping motor 21 within the handle 13, via a dog clutch 254.

The mechanism shown in FIG. 31 comprises a pair of spools 266 that contra-rotate on respective parallel longitudinal axes 268 by virtue of intermeshing gears 270. One of the spools 266 is driven directly by the dog clutch 254 and the other spool 266 is driven indirectly via the intermeshing gears 270. One spool 266, shown here on the left, reels in an end portion of the drive cable 26 as the other spool 266, shown here on the right, pays out the opposed end portion of the drive cable 26. The respective portions of the drive cable 26 pass through a set of guides 272 on their path to and from the shaft 242. It will be apparent that if drive to the dog clutch 254 is reversed, the operation of the spools 266 will also reverse to reverse the motion of the drive cable 26 and hence to reverse rotation the head 14.

Each spool 266 has a splined spindle or spigot 274 at a proximal end and a threaded spindle or spigot 276 at a distal end, both spigots 274, 276 extending longitudinally and being coaxial with the axis 268 of that spool 266. The splined spigots 274 are received in complementary splined sockets 278 that are integral with, or otherwise turn with, the gears 270. By virtue of their splined or keyed profiles, the spigots 274 and sockets 278 are in fixed angular relation but the spigots 274 can slide longitudinally in and out of the sockets 278.

Conversely, the male threaded spigots 276 are received in complementary female threaded sockets 280 that are fixed relative to the body 244. By virtue of their interlocking threads, rotation of the spigots 276 within the sockets 280 moves the spigots 276, and hence the remainder of the spools 266, longitudinally along the axes 268 relative to the sockets 280 and the body 244. This drives the aforementioned longitudinal movement of the splined spigots 274 within the splined sockets 278.

As the threads of the spigots 276 and sockets 280 associated with both spools 266 have the same handedness but the spools 266 contra-rotate, it will be apparent that the rotating spools 266 will move in opposite longitudinal directions as shown. This longitudinal movement avoids the drive cable 26 being wound in overlapping coils and therefore facilitates smooth and reliable winding and unwinding of the drive cable 26 onto and from the spools 266. Optionally, the spools 266 could have external spiral groove formations akin to a screw thread to guide the coils of the drive cable 26 during winding and unwinding.

Opposed longitudinal movement of the spools 266 also maintains a consistent gap between the set of guides 272 and the intersection of the drive cable 26 with the spool 266. This helps to maintain consistent tension in the drive cable 26, enabling the drive cable 26 to drive rotation of the head 14 reliably and faithfully without slipping or jumping. Optionally, as shown, a bias mechanism 282 biases the shaft 242 distally away from the body 244 of the probe assembly 12, which also has the effect of maintaining tension in the drive cable 26.

The opposed ends of the drive cable 26 are anchored in tie holes 284 on the respective spools 266. In this way, the drive cable 26 can be open-ended and so is simple to manufacture as it does not have to be in a continuous loop.

The mechanism shown in FIG. 31 will drive the head 14 of the probe assembly 12 to turn a set amount in each direction depending on the diameter of the spools 266 relative to the pulley groove 28 of the head 14, the number of coils of the drive cable 26 held by the spool 266, and the thread pitch of the spigots 276 and sockets 280. The mechanism can be set up in various ways with a combination of right- and left-hand threads, and rollers, belts or additional gears to achieve a similar effect. Optionally, the position or orientation of the spools 266 or of the set of guides 272 relative to the surrounding body 244 can be adjusted in a manner that will now be described with reference to FIG. 32, where like numerals are used for like features.

In FIG. 32, a single spool 266 driven directly via the dog clutch 254 about a longitudinal axis 268 carries a series of coils of a continuously-looped drive cable 26. The drive cable 26 is wound in at one end of the spool 266 and paid out at the other end of the spool 266. The coils of the drive cable 26 are forced to slide along the spool 266 to make space for additional coils to be wound onto the spool 266. Lubrication may be provided to assist that sliding movement.

Optionally, as shown, the spool 266 is shaped to help the drive cable 26 to slide along the spool 266 and to provide additional tension at the arrival and departure points where the drive cable 26 is wound onto and paid out from the spool 266 via the set of guides 272. This maintains tension in the drive cable 26 while allowing the coils of the drive cable 26 to slide more easily along the spool 266. In this example, the spool 266 has a concave, waisted hourglass profile that is relatively narrow between the arrival and departure points of the drive cable 26.

FIG. 32 shows other optional features, namely provisions for adjusting the position or orientation of the spool 266 and/or the set of guides 272 relative to the surrounding body 244 as noted above. Specifically, the spool 266 has distal and proximal spindles 286 embraced by respective bearing blocks 288. The bearing blocks 288 can be moved by screws 290 relative to the body 244 and to the set of guides 272 in directions transverse to the axis 268. Increasing the spacing between the spool 266 and the set of guides 272 increases tension in the looped drive cable 26.

In this example, the bearing blocks 288 can be moved independently, thus tilting the axis 268 relative to the body 244. This allows for differential adjustment of tension in the respective portions of the drive cable 26 upstream and downstream of the spool 266.

Another tension adjustment provision shown in FIG. 32 is a guide mount 292 that can be moved relative to the body 244 to move at least some of the set of guides 272 toward and away from the spool 266. In this example, the guide mount 292 is movable longitudinally, generally parallel to the axis 268 by a screw 290. However, the guide mount 292 could also, or alternatively, by moved transversely relative to the axis 268. Bias such as spring loading may also, or alternatively, be applied at the adjustment points exemplified by the screws 290. Further, although not shown in FIG. 32, provision such as the bias mechanism 282 of FIG. 31 could bias the shaft 242 distally away from the body 244 to increase tension in the drive cable 26.

The limited space available within the narrow shaft 242 of the probe 12 due to miniaturisation imposes constraints on drive systems that can generate torque sufficient to turn the head 14 about an axis perpendicular to the shaft 242. It is also challenging to minimise errors caused by any disparity between input and output at the proximal and distal ends of the endoscope 10 respectively. In other words, for a given input from a proximal drive mechanism, a change in the angular position of the head 14 must be reasonably calculable and predictable based on the position or movement of the drive mechanism.

To deal with this challenge, belt or cable drive systems may require a drive cable 26 to be looped multiple times around pulleys or spools at either end, like a capstan, to prevent slippage under high loads. Even so, there could still be a possibility of some slippage of the drive cable 26 relative to driving, or driven, pulleys or spools.

In a miniaturised system, space constraints impose a trade-off between the thickness of a drive cable 26 and the number of times the drive cable 26 can be looped, wrapped or turned around a pulley. Reducing the thickness of the drive cable 26 to increase the number of loops, wraps or turns that can be accommodated reduces its stiffness, which increases the risk and/or effect of a disparity between inputs and outputs. Also, as noted above, manufacture of a suitable closed-loop drive cable 26 can present design challenges.

To address these various challenges, FIGS. 33 and 34 illustrate a worm-gear mechanism that provides for torque to be transmitted directly to the head 14 within a shaft 242 of a probe 12 while also providing significant mechanical advantage. In this respect, the worm-gear mechanism comprises a spiral worm formation 294 at the distal end of a thin rod 296 extending within and generally parallel to the shaft 242. The rod 296 serves as a transmission shaft for transmitting torque from a drive, such as the aforementioned stepping motor 21, located proximally with respect to the shaft 242.

The worm-gear mechanism further comprises a worm gear 298 that is integral with, or attached to, the head 14 at the distal end of the shaft 242. The worm gear 298 comprises a series of part-spiral grooves 300 spaced circumferentially around, and angled acutely with respect to, the axis of rotation 22 of the head 14. The worm formation 294 turns around an axis that is generally tangential to the worm gear 298 and meshes with the grooves 300 to turn the worm gear 298 and the head 14 in response to rotation of the rod 296.

Positive mechanical engagement due to meshing between the worm formation 294 and the worm gear 298 reduces the possibility of slippage and so improves control over the angular position of the head 14. Also, it is advantageous for rotary drive to be transmitted along the length of the shaft 242 about an axis of rotation that extends along the shaft 242. This minimises losses, backlash and the effect of tolerances.

To facilitate miniaturisation, FIGS. 33 and 34 show two alterations to traditional worm gear designs. Rather than being machined into a shaft, the worm formation 294 comprises a slender wire that is formed into a coiled or helical shape at the distal end of the rod 296. The worm formation 294 may be formed integrally with, or may be attached to, the rod 296. Conversely, the worm gear 298 is created with a pattern of cut-out grooves 300 complementary to the helical shape of the worm formation 294 to allow smooth transmission of rotary drive from the rod 296.

The axis of rotation of the worm formation 294 cannot be coaxial with the central axis of the shaft 242, which presents challenges to miniaturisation. There are also constraints on the helical shape of the worm formation 294 in contact with the worm gear 298. The rod 296 may therefore be reinforced to increase its structural integrity.

FIG. 35 shows that a system combining belt-drive and gear-drive principles may provide a solution for transmitting torque while minimising strain on different parts of the system. Here, a rod 296 serves as a transmission shaft that can lie along a central axis of the probe shaft 242 and can be turned about that axis to transmit torque toward the distal end of the shaft 242. Near the distal end of the shaft 242, meshed bevel gears 302 turn the axis of rotation by 90°. A pulley 304 that turns with the second bevel gear 302 is aligned with the pulley groove 28 of the head 14 to transmit drive to the head 14 via a short continuous-loop drive cable 26. It should be noted that in this arrangement, the cleaning module or wiper blade 30 would be located between the pulley 304 and the rotating head 14.

The arrangement of FIG. 35 may not benefit from the same mechanical advantage as the worm gear design shown in FIGS. 33 and 34. However, locating the rod 296 on the central axis of the probe shaft 242 allows the rod 296 to be thicker and more rigid, therefore potentially reducing strain arising from torsion of the rod 296. Also, the short span between the pulley 304 and the pulley groove 28 reduces strain on the drive cable 26 and therefore reduces disparities between inputs and outputs. That short span also makes it easier to manufacture a suitable closed-loop drive cable 26.

Turning now to FIG. 36, the endoscope 10 shown schematically here requires white light illumination to be transmitted from a high-brightness source 27, for example contained in the handle 13 at the proximal end of the endoscope 10, along a light guide 32 to the distal tip of the probe 12 and from there to the head 14. Simultaneously, data carried by an optical signal is transmitted between electronic components in the form of optical transceivers in the handle 13 and the head 14. Due to the spatial constraints in the miniaturised endoscope 10, these signals are transmitted in the same light guide 32 extending along the probe 12 and, proximally, into the body 244 of the probe 12.

The arrangement shown in FIG. 36 combines these two optical signals, namely illumination and data, in the handle 13 so that light can be transmitted efficiently to the head 14 across an interface 306 between the handle 13 and the body 244. The solution shown in FIG. 36 also separates the data signal returning from the head 14 to the handle 13 so that the data signal may be processed further electronically. As the arrangement combines and separates optical signals, it may be described as an optical multiplexer. FIG. 36 shows one possible design for such an optical multiplexer.

The arrangement of FIG. 36 comprises an optical multiplexer element 308 placed in the handle 13 of the endoscope 10. The handle 13 is intended to be a reusable component of the system and to connect directly to the body 244 of the probe 12, being a disposable or partially recyclable component of the system.

The element 308 is so positioned in the handle 13 that the distal end of the element 308 confronts and communicates optically with the proximal end of the light guide 32 in the body 244, when the handle 13 is engaged with the body 244. Thus, the mechanical configuration of the handle 13 is such that once it mates with the body 244 of the probe 12, the element 308 is aligned longitudinally and laterally with the light guide 32 to minimise insertion losses across the interface 306.

The element 308 is made from a transparent material, such as glass or a suitable polymer, with high transmission properties in visible and near-infrared wavelengths. The element 308 has a 45° chamfer or bevel 310 at its proximal end, coated with a short-pass dichroic coating. The coating of the bevel 310 is transparent to light with a wavelength below a cut-off level, which may be defined in this case as a boundary between visible and near-infrared light. Wavelengths above that cut-off level, hence being near-infrared light, are reflected by the coating of the bevel 310.

In the arrangement of FIG. 36, light in the visible wavelength range from an illumination source 27 passes through the coating of the bevel 310 and so is guided through the element 308 and into the light guide 32. Meanwhile, light in the near-infrared range emitted by a transceiver 312 is reflected by the coating of the bevel 310 and so is also guided through the element 308 and into the light guide 32. Similarly, data signals returning along the light guide 32 from the head 14 of the endoscope 10 are reflected at the coated surface of the bevel 310 to be directed onto the transceiver 312.

FIG. 36 shows optional lenses 314 that may be used to optimise coupling of light into the element 308 and, in the case of the data signal, collection of emitted light to be focused onto the transceiver 312.

In the variant shown in FIG. 37, the light sources being the illumination source 27 and the transceiver 312 are still located in the handle 13. However, the multiplexer is not a separate element forming part of the handle 13 but is instead a chamfered or bevelled proximal end part of the light guide 32 itself. In this variant, insertion losses across the interface 306 are eliminated.

In this example, the proximal end part of the light guide 32 defining the multiplexer protrudes proximally from the body 244 and is protected by an external housing 316 of the body 244. A 45° bevel 310 at the proximal end of the light guide 32 is again coated with a short-pass dichroic coating and has the same functions as described above in relation to FIG. 36.

When the handle 13 is engaged with the body 244, the housing 316 is received in a complementary socket 318 of the handle 13 as a male/female mechanical coupling. The socket 318 supports the lenses 314 and the housing 316 has windows or apertures that are then aligned with or opposed to the lenses 314 for optical communication of the light guide 32 with the illumination source 27 and the transceiver 312 via the bevel 310.

In another variant, also applicable to FIG. 36, the dichroic coating of the bevel 310 may be replaced by a polarising beam splitter. In that case, the light signals of illumination and data are linearly polarised orthogonally to each other. One polarisation state, associated with data, is reflected by the splitter to and from the transceiver 312 and the other polarisation state, associated with illumination, passes straight through the splitter from the illumination source 27.

The source of white light serving as the illumination source 27 may be a light-emitting diode (LED), RGB laser, laser-pumped phosphor source or other equivalent broadband, visible light emitting source.

Moving on now to FIG. 38, visualisation of certain structures during surgery—for example, between blood vessels and tissue parenchyma—can be challenging due to low contrast under standard white light illumination. Visualisation may be improved by exploiting the optical properties of the endogenous pigments in the body and illuminating the tissue with light at wavelengths enabling greater contrast to be observed. For example, shorter visible wavelengths in the blue or green range are more strongly absorbed by haemoglobin in blood than red. Consequently, illuminating tissue with such wavelengths would allow enhancement of blood-rich features from the background.

Contrast may also be enhanced by introducing a dye, either topically or intravenously, to provide exogenous contrast enhancement. This may be achieved either passively by flowing dye to, diffusing dye or otherwise staining a tissue of interest, or actively using molecular targeting. In one variant, the dye could be fluorescent and emit light at a particular wavelength when a specific excitation wavelength is incident on it. In another variant, the dye could be a pigment that absorbs light of a specific wavelength or wavelength range.

In this case, the illumination source must be controlled so that the contrast enhancement signal (for example, the fluorescence emission or the absorption signal) is not contaminated or washed out by other overlapping wavelengths used for standard illumination. One method is to separate, temporally, the signal wavelength representing the peak of the fluorescence excitation light or pigment absorption from the standard white light illumination source by alternately switching them on and off in a strobe manner, as shown at the top of FIG. 38. If this strobing is done synchronously with image acquisition, as also shown in FIG. 38, the video feed may be processed subsequently to select frames showing either standard white light illumination, contrast-enhanced illumination, or a combination with contrast enhancement overlaid on the standard illuminated background. Simulated views under these three scenarios, as seen by a surgeon on a system display, are shown at the bottom of FIG. 38.

Illumination requires multiple wavelength channels for both endogenous and exogenous contrast enhancement. Such channels include those capable of providing standard white light illumination and, in the case of exogenous contrast agents, an excitation wavelength. With reference to FIG. 39, an optical multiplexer 320 combines these multiple wavelengths into a single light guide 32 that subsequently guides this light to the head 14 of the endoscope 10 to illuminate the tissue.

Conveniently, the multiplexer 320 can be incorporated in the handle 13 of the endoscope 10 as shown in FIG. 39. In this example, the multiplexer 320 is a proximal extension of the light guide 32 in a manner akin to the arrangement shown in FIG. 37, that extension being contained within a housing 316 that protrudes from the body 244 and is received in a socket 318 in the handle 13. In other examples, the multiplexer 320 could comprise a separate element in optical communication with the light guide 32 across the interface 306, like the element 308 shown in FIG. 36

FIG. 39 shows an optical transceiver 312 used to communicate optical data to and from the head 14 at the distal end of the endoscope 10. Further emitters 322 are positioned in axial succession along the multiplexer 320, in this case in addition to a white light source 27 mounted axially at the end of the multiplexer. The emitters 322 and the white light source 27 emit light at respective different wavelengths.

In this example, the various light sources being the transceiver 312, the emitters 322 and the white light source 27 communicate with the light guide 32 via respective lenses 314. As before, the lenses 314 optimise coupling of light into the light guide 32 and, in the case of the data signal, collection of emitted light to be focused onto the transceiver 312.

The transceiver 312 and the emitters 322 are in axial alignment with respective short-pass dichroic filters 324 in the light guide 32, each angled at 45° to the longitudinal axis of the light guide 32. Thus, in this variant, the input ports of the multiplexer 320 are formed by stacking multiple guiding elements separated by the filters 324. The light sources are arranged, from top to bottom as illustrated or in distal succession, in order of increasing wavelength from the white light source 27 through the emitters 322 to the transceiver 312. The filters 324 are designed with cut-off wavelengths between the wavelength ranges of each of those sources.

Further light sources may be used simply by stacking dichroic filters 324 additional to those illustrated in FIG. 39. As before, the light sources used may be light-emitting diodes (LEDs), lasers or equivalent.

FIG. 40 shows a possible arrangement for the light guide 40 within the head 14 as featured in preceding embodiments. To recap, illumination and optical data signals combined using an optical multiplexer/demultiplexer in the handle 13, like the arrangements shown in FIGS. 36, 37 and 39, travel down the light guide 32 of the probe 12 until they are reflected into the head 14 by a 90° prism 34. The prism 34 directs the light along the rotational axis 22 of the head 14 and into the light guide 40 illustrated in FIG. 40.

The light guide 40 serves as an optical demultiplexer by separating illumination from the optical data signals, directing the illumination to a light emitter 18 and the optical data signals to an optical transceiver implemented by a transmitting and receiving LED 174 coupled to electronics of the camera module 16. Conversely, the light guide 40 also serves as an optical multiplexer for receiving optical data signals emitted by the LED 174 and coupling them back into the light guide 32, to be transmitted to the optical transceiver 312 via the optical multiplexer/demultiplexer in the handle 13.

In the variant shown in FIG. 40, two dichroic filters 326, 328 intersect with each other orthogonally in an ‘X’ formation centred on the rotational axis 22 of the head 14, each filter 326, 328 being oriented at 45° to light entering and leaving the light guide 40. The filter 326, shown as a dashed line, is a short-pass filter that is transparent to visible light and reflective to near-infrared light, whereas the filter 328, shown as a solid line, is a long-pass filter that is transparent to near-infrared light and reflective to visible light. This arrangement achieves the required separation of incoming illumination, shown in solid lines, from incoming and outgoing data signals, shown in dashed lines.

Detail A in FIG. 40 shows how the intersecting dichroic filters 326, 328 may be assembled from four separate optical elements 330, 332. A pair of the elements 330 aligned with each other and with the light emitter 18 and the LED 174 each have a tapered tip defined by facets 334 that converge at 90° to each other. The other pair of elements 332 oriented at 90° to the elements 330 have similarly tapered tips that engage laterally with and between the elements 330 to complete the light guide 40.

The elements 332 are uncoated but the facets 334 of each element 330 are coated to define the desired characteristics of the filters 326, 328. Specifically, one facet 334 of each element 330, represented here by a dashed line, is coated to define the short-pass characteristic of the filter 326. The other facet 334 of each element 330, represented here by a solid line, is coated to define the long-pass characteristic of the filter 328.

When the elements 330 are brought together tip-to-tip, their facets 334 combine to define the filters 326, 328. In this respect, it will be noted that the short-pass coated facets 334 and the long-pass coated facets 334 of the elements 330 are in diagonal opposition about the abutting tips of the elements 330.

In another variant, the light guide 40 shown in FIG. 40 could be used to separate wavebands other than visible and near infrared. Any two wavebands may be selected for the design of the illumination and data channels, with the choice being implemented in the optical properties of the dichroic filters 326, 328. These coatings may be long- or short-pass, with cut-on/off wavelength at varying positions, or they may have a bandpass structure to transmit or reflect, selectively, narrow or broad wavelength ranges and multiple non-contiguous bands.

In a further variant, as mentioned above in relation to FIGS. 36 and 37, the dichroic filters may be replaced by a polarising beam splitter acting on orthogonally polarised light used for the data and illumination channels.

Turning finally to FIGS. 41 and 42, free 360° rotation of the head 14 requires circuitry to be electrically isolated and therefore for data and power to be transmitted wirelessly. The requirement of wireless power transmission in such a confined space presents challenges where the data comprises a video signal that is susceptible to distortion. Consequently, a solution is required that can mitigate power and data losses while allowing 360° rotation of the head 14.

FIG. 41 is a block diagram that outlines this solution as a system overview, whereas FIG. 42 shows details of the system shown in FIG. 41. The system shown in FIGS. 41 and 42 is divided into two parts, namely a disposable part and a reusable part. The probe 12 and the head 14 are in the disposable part of the system whereas the handle 13 and the camera control system or CCU 15 are in the reusable part of the system.

In this solution, a clock signal and power are transmitted together on a single channel using a superimposed signal like that shown in FIG. 27. In the reusable part of the system, a high-frequency clock signal is generated to be used as a power transfer clock. This signal requires a phase lock condition with a signal provided by an image sensor companion chip 230, such as at 24 MHz.

On the receiver side, in the head 14 of the disposable part of the system, a digital frequency divider is required to recover the clock signal at the correct frequency of 24 MHz. A simple frequency divider can be used if the frequency of the power clock signal is an integral multiple of the 24 MHz clock. The frequency divider is simpler if the multiplier is a power of two, for example two, four or eight. For simplicity, the solution shown in FIGS. 41 and 42 uses four as the multiplier.

For reliable data signal transfer, the reconstructed 24 MHz clock signal in the head 14 must have an equal phase relation to the clock signal generated outside the head 14. To solve this problem, the system provides a phase adjustment.

In this example, the image sensor 48 of the head 14 uses a 24 MHz clock signal that is generated in the image sensor companion chip 230. Power transfer can be effected by an inductive coupling. The power circuit may, for example, employ a sinusoidal (‘sinus’) signal with a frequency of 96 MHz (being four times the clock frequency of 24 MHz in this example) to allow the use of small and compact planar coils 182 for power transmission. As noted previously, such coils 182 could be made using a two-layer flex substrate.

Due to the high frequency of the power signal, the SPI data transfer method described in previous embodiments may not be appropriate. For this purpose, the optical channel video data signal path is reused in a reverse direction for the SPI control signal.

As in the camera control unit 15 of the embodiment of FIG. 26, an image signal processing chip 224 responsive to an embedded computer 226 drives an SPI controller 228 implemented on an image sensor companion chip 230. In this case, however, the SPI controller 228 feeds SPI write data to the optical transceiver 312.

The image sensor companion chip 230 also implements a clock generator 232 that generates the 24 MHz signal. The 24 MHz signal is multiplied fourfold to 96 MHz in a frequency multiplier 336 and fed to a power and clock sinus filter driver 338 before onward wireless transmission of power and clock signals to the head 14 through the inductive link at 182, across the interface between the probe 12 and the head 14.

In the head 14, a power harvester chip 340 receives power from the inductive link at 182 and supplies that power to a power regulator 33. The power regulator 33 regulates the power to drive the image sensor 48 and supporting circuitry. A filter 216 filters the clock signal from the power signal. The clock signal is then divided by four in a digital frequency divider 218 to control the image sensor 48, hence returning to a frequency of 24 MHz.

In the optical channel video data signal path, a sync separator 342 receives SPI data from the optical transceiver 312. A clock phase alignment detector 344 receives inputs from the sync separator 342 and the clock generator 232 of the companion chip 230 to apply phase control to the frequency multiplier 336.

As in FIG. 26, an optical demodulator 236 receives and demodulates signals from the optical transceiver 312 and feeds its output to the image signal processing chip 224 via an analog-to-digital converter 238 and a parallel mobile industry processor interface 240.

Many other variations are possible within the inventive concept. For example, data communication and power transmission between parts of a surgical instrument could, in principle, be achieved by wireless techniques other than those described, such as magnetic resonance communication. However, electrical contacts on, or conductors between, the outer surfaces of adjoining capsules, modules or components could potentially be used for this purpose.

Data communication and power transmission in an instrument may be effected by daisy-chaining between successive capsules, modules or encapsulated components of the instrument. Thus, each capsule, module or component in the daisy-chain arrangement has a means of receiving data or power from an external source and/or a means of transmitting or conveying data or power to an external receiver. Such daisy-chain features are exemplified in this specification by the provisions for receiving light into the head to power the camera module and by the provisions for transmitting data from the camera module to an external receiver. In the case of RF communication, daisy-chaining is advantageous because the short transmission distance means that power can be kept low, making it easier to provide sufficient electrical energy and ensuring that RF radiation does not penetrate deep into the patient's body.

In some embodiments of the invention, external elements of the endoscope may be made from biodegradable materials, thus further reducing waste that would otherwise be sent for recycling or incineration. This is most suitable for structural parts such as handles. The biodegradable parts can form part of a capsule and be disassembled as previously described. Alternatively, a second internal layer can form a hermetic seal. In that case, the second internal layer could be of a non-structural nature and could be supported and protected by an outer biodegradable layer.

1-37. (canceled)

38. A medical scope, comprising:
an elongate body;
an imaging head supported by and movable relative to a mount of the body about an axis of rotation extending from the mount, the imaging head including a light emitter for illuminating a field of view of the imaging head; and
a light path extending from the body parallel to the axis of rotation and through the imaging head to the light emitter, the light path including a light inlet of the imaging head opposed to a light outlet of the body.

39. The scope of claim 38, wherein the light inlet and the light outlet are mutually opposed across a mounting interface at which the imaging head is movably attached to the mount.

40. The scope of claim 38, wherein the imaging head comprises:
an image sensor; and
a photovoltaic generator arranged for impingement of light from the body to generate electrical power for the image sensor.

41. The scope of claim 40, further comprising a data transmitter powered by the photovoltaic generator to transmit image data from the image sensor to a receiver outside the imaging head.

42. The scope of claim 40, wherein the imaging head further comprises a light guide in the light path, configured to direct a portion of light received from the body onto the photovoltaic generator and another portion of light received from the body to the light emitter.

43. The scope of claim 42, wherein the light inlet and the light outlet are disposed on said axis of rotation, and the light guide is disposed between the mount and the image sensor in a direction parallel to that axis.

44. The scope of claim 43, wherein the light emitter is also disposed between the mount and the image sensor in a direction parallel to the axis of rotation, at a position spaced radially from the axis of rotation.

45. The scope of claim 42, wherein the light guide comprises a filter configured to divide the light received from the body into said portions to be directed onto the photovoltaic generator and toward the light emitter.

46. The scope of claim 40, wherein the imaging head comprises encapsulation around the image sensor, that encapsulation including a disassembly interface at which the image sensor is preferentially separable from the light emitter.

47. The scope of claim 46, wherein the disassembly interface comprises at least one point of weakness in the encapsulation.

48. The scope of claim 38, wherein the imaging head is cantilevered from the mount.

49. A method of illuminating a field of view of an imaging head of a medical scope, which head is movable relative to a supporting body of the scope about an axis of rotation extending from the body, the method comprising: conveying light along a light path from a light outlet of the body parallel to said axis of rotation to a light inlet of the imaging head and then through the imaging head to be emitted from the imaging head.

50. The method of claim 49, comprising powering an image sensor of the imaging head with electrical power generated within the imaging head from at least a portion of the light conveyed along the light path.

51. The method of claim 50, comprising filtering light travelling on the light path within the imaging head to separate that light into components for power generation and for illumination, respectively.

52. The method of claim 51, comprising varying at least one of said components of the light relative to another of said components of the light for independent adjustment of power generation and/or illumination.

53. A medical scope, comprising:
an imaging head supported by and movable relative to a body about an axis of rotation extending from the body, the imaging head including an image sensor and a light emitter for illuminating a field of view of the image sensor;
a light path extending from the body parallel to the axis of rotation to the light emitter, the light path including a light inlet of the imaging head opposed to a light outlet of the body across a wireless interface between the imaging head and the body; and
a light transmitter in the imaging head, the light transmitter being coupled to the light path to transmit image data from the image sensor to the body across the interface.

54. The scope of claim 53, wherein the light inlet and the light outlet are mutually opposed across a mount at which the imaging head is movably attached to the body.

55. The scope of claim 53, wherein the light inlet and the light outlet are disposed on said axis of rotation about which the imaging head can turn relative to the body.

56. The scope of claim 55, wherein the light emitter is disposed between the interface and the image sensor in a direction parallel to the axis of rotation, at a position spaced radially from the axis of rotation.

57. The scope of claim 53, wherein the interface further comprises a wireless power transmission coupling effecting transmission of power from the body to the image sensor.

58. The scope of claim 57, wherein the power transmission coupling comprises mutually-opposed coils, a first of those coils surrounding the light inlet of the imaging head and a second of those coils surrounding the light outlet of the body.

59. The scope of claim 53, wherein the imaging head further comprises a photovoltaic generator for powering the image sensor from light transmitted across the interface.

60. The scope of claim 53, wherein the imaging head further comprises a filter in the light path, configured to block reflected light from the light emitter and to pass light transmitted from the light transmitter.

61. The scope of claim 53, further comprising a light source outside the imaging head and coupled to the light path to provide illuminating light to the light emitter, wherein the light source and the image sensor are synchronised such that the light source is on when the image sensor is capturing an image frame and the light source is off between image frames while the light transmitter transmits imaging data from the image sensor.

62. The scope of claim 53, further comprising an image sensor controller located outside the imaging head and being arranged to convey a control signal from the image sensor controller across the interface to the image sensor.

63. The scope of claim 62, wherein the control signal comprises a clock signal multiplexed with a write signal.

64. The scope of claim 62, wherein the image sensor controller is connected to a wireless power transmission coupling of the interface to convey the control signal to the image sensor through that coupling.

65. The scope of claim 62, wherein the image sensor controller drives an LED to convey the control signal to the image sensor as an optical control signal.

66. The scope of claim 65, wherein the light transmitter in the imaging head is responsive to the optical control signal.

67. A method of operating a scope that comprises an imaging head movable relative to a supporting body about an axis of rotation extending from the body, the method comprising:
illuminating a field of view of the imaging head by conveying light along a light path parallel to said axis of rotation across a wireless interface from the body to the imaging head; and
transmitting image data optically along the light path across the interface from the imaging head to the body.
